(19) United States
(12) Patent Application Publication
(10) Pub. No.: US 2014/0178367 A1
(43) Pub. Date: Jun. 26, 2014

(54) Methods of Treating Inflammatory Diseases by Targeting the Chemoattractant Cytokine Receptor 2 (CCR2) or Chemokine (C-C motif) Ligand 2 (CCL2)

(71) Applicants: Howard Weiner, Brookline, MA (US); Oleg Butovsky, Boston, MA (US)

(72) Inventors: Howard Weiner, Brookline, MA (US); Oleg Butovsky, Boston, MA (US)

(73) Assignee: THE Brigham and Women's Hospital, Inc., Boston, MA (US)

(21) Appl. No.: 14/110,012

(22) PCT Filed: Apr. 5, 2012
(86) PCT No.: PCT/US2012/032354

§ 371 Date: Dec. 18, 2013

Related U.S. Application Data
(60) Provisional application No. 61/472,996, filed on Apr. 7, 2011.

(51) Int. Cl. C07K 16/28; C07K 16/24

(52) U.S. Cl. CPC C07K 16/2866; C07K 16/24
USPC 424/133.1; 424/172.1; 424/158.1; 424/143.1; 424/145.1

(57) ABSTRACT

Methods of treating inflammatory diseases, e.g., diseases associated with inflammatory CD14+/CD16− monocytes, e.g., amyotrophic lateral sclerosis (ALS), stroke, and glaucoma, using compounds such as small molecules and antibodies that target CCR2 or CCL2.

### CLAIM OF PRIORITY

This application claims the benefit of U.S. Provisional Patent Application Ser. No. 61/472,996, filed on Apr. 7, 2011. The entire contents of the foregoing are hereby incorporated by reference.

### FEDERALLY SPONSORED RESEARCH OR DEVELOPMENT

This invention was made with Government support under Grant No. AG027437 awarded by the National Institutes of Health. The Government has certain rights in the invention.

### TECHNICAL FIELD

This invention relates to methods of treating inflammatory diseases, e.g., diseases associated with inflammatory CD14+/CD16− monocytes, e.g., amyotrophic lateral sclerosis (ALS), multiple sclerosis, stroke, and glaucoma, using compounds such as small molecules and antibodies that target the Chemoattractant Cytokine Receptor 2 (CCR2) or Chemokine (C—C motif) Ligand 2 (CCL2).

### BACKGROUND

During inflammation, monocytes give rise to monocyte-derived dendritic cells (DCs), including tumor necrosis factor (TNF) and inducible nitric oxide synthase (iNOS)—producing dendritic cells (TipDCs), and inflammatory macrophages.

### SUMMARY

The present invention is based, at least in part, on the discovery that systemic treatment with an agent, such as an antibody or small molecule, targeting a specific population of immune cells (in humans, CD14⁺/CD16⁻/CCR2⁺ monocytes) leads to attenuation of clinical score in ALS mice, decreased necrotic lesions in a mouse model of brain stroke, and protection of retinal ganglion cells in the eye of mouse model of glaucoma.

In one aspect, the invention provides methods for treating subjects suffering from a condition selected from the group consisting of amyotrophic lateral sclerosis (ALS), stroke, and glaucoma, by administering to the subject an effective amount of a compound that binds to and inhibits Chemoattractant Cytokine Receptor 2 (CCR2) or Chemokine (C—C motif) Ligand 2 (CCL2).

In one aspect, the invention provides methods for reducing inflammation in a subject suffering from a condition selected from the group consisting of ALS, stroke, and glaucoma, by administering to the subject an effective amount of a compound that binds to and inhibits CCR2 or CCL2.

In some embodiments, the subject is suffering from ALS; in some embodiments, the subject is suffering from glaucoma; in some embodiments, the subject is suffering from a stroke.

In some embodiments, the compound is a small molecule inhibitor of CCR2 or

CCL2.

In some embodiments, the compound is an antibody or antigenic fragment thereof that binds to CCR2 or CCL2. In some embodiments, the antibody is a monoclonal antibody or CCR2- or CCL2-binding fragment thereof In some embodiments, the antibody is a human, humanized or chimeric antibody.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-B. Reciprocal expression of CD39 and Ly6C in CNS-resident microglia and inflammatory monocytes in healthy adult mice. (A) qRT-PCR of Ly6C and CD39 expression in adult microglia (CD11b+/CD45Low) and CD11b+/Ly6C− and CD11b+/Ly6C+ sorted monocyte subsets from PBMC, spleen, and BM from naïve adult C57BL/6 mice. Expression levels were normalized to GAPDH. (B) Cytometry histograms show intensity MFI of surface expression of CD39 and Ly6C in organ-specific CD11b-gated cells compared to isotype control (open histograms) from naïve B6 mice. Each histogram panel represents a pool of 5 mice. The data shown are representative of two for A and five for B independent experiments.

FIGS. 2A-E. Reciprocal expression of CD39 and Ly6C in CNS-resident microglia and BM-derived monocytes in SOD1G93A chimeric mice. SOD1G93A and WT mice were transplanted with BM cells from CX3CR1-GFP+/− Spinal cords were taken at age of 145d (end-stage). (A) GFP+ recruited IBA1+ monocytes in lumbar spinal cord and throughout the affected regions of these two types of chimeric mice (as indicated). (B) Confocal images of GFP+monocytes (IBA1+/GFP+; smaller arrows) and resident microglia (IBA1+/GFP−; larger arrow at top right) in ventral horns of WT (non-Tg)- and SOD1G93A-chimeric mice. Representative confocal images of 5-6 mice per group. (C) Quantitative analysis showing the kinetics of BM-derived CX3CR1-GFP monocytes recruited into spinal cords during disease at pre-symptomatic (90d), early-symptomatic (120d) and late-symptomatic (145d) in SOD1G93A chimera mice. (D) Expansion of recruited Ly6CHi/CX3CR-GFPLow monocytes subset in the spinal cord of SOD1G93A during disease progression. (E)

FACS analysis of CD39 and Ly6C expression in spinal cord-derived populations of microglia (MG) and peripheral monocytes (PMs) isolated from WT (non-Tg)- and SOD1G93A-chimera mice at the early onset of the disease. Note, CD11b+/GFP+-gated peripheral monocytes do not express CD39 and are positive for Ly6C, whereas all resident microglia express CD39 and negative for Ly6C. Each panel represents a pool of 4-5 mice. The data shown are representative of two independent experiments.

FIGS. 3A-E. Ly6CHi monocytes recruited to the spinal cord with disease progression in SOD1G93A mice. (A) FACS analysis of isolated spinal cord and brain-derived mononuclear cells for CD11b, CD39 and Ly6C at 135d in SOD1G93A mice. Cells were gated using AnnexinV and 7-AAD to eliminate apoptotic and necrotic cells. (B) Proportional increase in inflammatory monocytes (black) and myeloid cells (gray) and decrease in CD39+ resident microglia (white) to total CD11b+ cells in the spinal cord of SOD1G93A mice. At 120d and 135d, respectively, the proportion of Ly6C monocytes and myeloid cells significantly increased compared with the age 135d SOD1WT (P<0.01 and P<0.001, respectively), whereas CD39+ microglia significantly decreased (P<0.01 and P<0.001, respectively). No contribution of myeloid subsets was detected in brains of SOD1G93A mice. The data represent mean±SEM from three experiments with pool of four or five mice per group. (C) Expansion of Ly6C monocytes from Ly6CLow to Ly6CHi during disease progression in the spinal cord. Note, mean fluorescence intensity (MFI) of Ly6C is increased during disease progression on recruited monocytes but not on resident microglia (G1 and G2, respectively). (D) G1 [CD11b+/Ly6C+]− and G2 [CD11b+/Ly6C−]-gated population (indicted in C). Note, CD11b+/Ly6C+ cells do not co-express CD39. Each panel represents a pool of 5 mice. The data shown are representative of three independent experiments. (E) qRT-PCR analysis of CCR2 and CCL2 mRNA expression in CD39+ microglia and Ly6CHi monocytes sorted by flow cytometry from SOD1WT and SOD1G93A mice at the disease onset and end-stage. Total RNA was isolated and pooled from five mice of each cell population. Expression levels were normalized to GAPDH. Representative of two independent experiments.

FIGS. 4A-F. Systemic treatment with anti-Ly6C mAb antibody improves body-weight maintenance, delays disease onset and extends survival in SOD1 mice. SOD1^{G93A} mice were treated intraperitoneally (ip) with IgG2a (IC; isotype control 100 μg, n=11) or anti-Ly6C mAb with 10 μg (n=6), and anti-Ly6C mAb with 100 μg (n=11) each second day starting at the onset of the disease. Onset of symptoms was defined by the peak of the weight curve and visible signs of muscle weakness. (A) Kaplan-Meir analysis of the probability of surviving of SOD1^{G93A} as function of age. Mantel-Cox's F-test comparison showed groups treated with isotype control (IC) vs. anti-Ly6C 100 μg (P=0.0097) or 10 μg (P=0.1187). (B) Time-to-event analysis for disease neurologic onset (neurological severity score of 2). Disease onset was significantly delayed (P=0.0127) by anti-Ly6C (100 μg) treatment. (C) Duration of an early disease phase (from onset to 5% weight loss) and a later disease phase (from 5% weight loss to end stage). Early and late symptomatic phases of disease were significantly delayed (P<0.0001 and P<0.05, respectively) by anti-Ly6C (100 μg) treatment, but not with 10 ug of anti-Ly6C (data not shown). Statistical analysis by one-way ANOVA. (D) Mean rotorod performance (±SEM) of IC- and anti-Ly6C-treated groups as a function of age. *P<0.05; **P<0.01 and ***P<0.001 compared IC to anti-Ly6C (100 μg) groups by factorial ANOVA and Fisher's LSD post-hoc test. (E) Weight loss plotted for IC-treated and abti-Ly6C (100 μg)-treated groups (*P<0.05; **, P<0.01; ***,P,0.001; 2-way ANOVA, Bonferroni post-test). (F) Cumulative results of statistical analysis of IC-treated and anti-Ly6C (100 μg)-treated groups.

FIGS. 5A-B. Ly6C treatment affects the phenotype of Ly6C^{Hi} monocytes in the spinal cord and spleen of SOD1 mice. SOD1^{G93A} mice were treated as in FIG. 4. After one month of treatment (120d of age), spleen- and spinal cord-derived CD11b⁻/Ly6C^{Hi} sorted cells were analyzed. (A) Cytokine profile of spleen-derived CD11b⁻/Ly6C^{Hi} cells in IC- and anti-Ly6C-treated SOD1^{G93A} mice. (B) Cytokine profile of spinal cord-derived CD11b⁺/Ly6C^{Hi} cells in IC- and anti-Ly6C-treated SOD1^{G93A} mice. Expression levels were normalized to GAPDH. Bars represent data from 3 pooled experiments, each with 3-6 mice. Error bars represent mean±SEM (**, P<0.01; ***, P,0.001; Two-tailed t-test).

FIGS. 6AE. Ly6C-treatment lowers the frequency of CD169⁻ and Ly6C⁺ monocytes and attenuates neuronal and CNS-resident microglial loss in the spinal cord of SOD1 mice. SOD1^{G93A} mice were treated as in FIG. 4. (A) FACS analysis of Ly6C⁺ monocytes in the spinal cord of anti-Ly6C-treated SOD1^{G93A} mice compared to IC group 30 days post-treatment. Pool of 5 mice is shown. (B) Significantly reduced proportion of Ly6C⁺ monocytes and increased number of CD39⁺ microglia out of CD11b⁻ cells 50 days after anti-Ly6C treatment. (C) Significant reduction of CD11b⁻/CD169⁺ monocytes was detected after 50 days of anti-Ly6C treatment. (D) Representative confocal images stained for NeuN (green; neurons), IBA1 (blue; myeloid cells) and CD169 (recruited monocytes; red) of whole mount lumbar axial sections of spinal cords from IC and Ly6C-treated mice at the end-stage (140d old). Boxed areas showed inserts at high magnification. (E) Quantitation of neurons (NeuN⁺), and recruited monocytes (IBA1⁺/CD169⁺) in ventral and dorsal horns in the spinal cord of SOD1^{G93A} mice treated with IC or anti-Ly6C mAbs (n=6-8 per group). Two-way ANOVA, Bonferroni post-tests. Similar Representavie of 2 experiments. Bars show data from one representative experiment (n=5 mice per group). Error bars ±SEM (P value by t test).

FIGS. 7A-C. CD169 expression in blood monocytes and spinal cord of ALS patients. (A) Representative (n=4) flow cytometry analysis of CD169 surface expression on CD14+ monocytes in normal subject and ALS patient. CD14+ gated cells were defined out of the population of live cells using AnnexinV and 7-AAD to eliminate apoptotic and necrotic cells. (B) CD14-gated cells were analyzed for co-expression of CD169. Significantly, higher percentage of CD169+/CD14+ cells was seen in ALS patients compared to the normal subjects. (C) Representative confocal images stained for NeuN (top panel; neurons), IBA1 (bottom right panel; myeloid cells) and CD169 (recruited monocytes; bottom left panel) in lumbar axial sections from ALS subject. Boxed areas on bottom panels show separate confocal lasers for CD169+ and IBA1+ cells (small arrows in lower panels).

FIGS. 8A-D. EAE progression is associated with indigenous microglia (4D4) loss and reciprocal increase in peripheral Ly6C^{Hi} inflammatory monocytes in the CNS. (A) FACS analysis of CNS-derived mononuclear cells from naïve and C57/B6 EAE-mice at presymptomatic (5d), onset (10d), peak (14-16d), early recovery (21d) and late recovery (28d) stages of the disease. CD11b⁺ cells analyzed for both 4D4 (upper panels) and 6C3 (bottom panels) expression. (B) EAE clinical score. (C) Statistical analysis of [CD11b⁺]-gated cells analyzed for 4D4 and 6C3 expression. (D) WB analysis of brain and spinal cord of EAE-mice at indicated stage of the disease.

FIGS. 9A-B. Recruitment of GFP+ BM-monocytes associated with 4D4+ indigenous microglia loss in EAE chimeric mice. C57/B6 mice at age of 8 weeks were transplanted with BM cells from transgenic mice expressing GFP under CX3CR1 promoter (See FIG. 13). 2 month later, the mice were vaccinated with MOG to induce EAE. Axial sections of spinal cords were taken at different stages of the disease, as indicated. (A) High-power confocal images show analyzed areas of ventral horn of the spinal cords stained for 4D4 (red in original, indigenous microglia), NeuN (blue, neurons) and GFP+ (green in original, BM-derived monocytes) (B) Low-power representative confocal images of the spinal cords stained for 4D4 (red in original), IBA1 (blue in original, microglia/monocytes) and GFP (peripheral recruited monocytes). Inserts showed high-power representative confocal images of changes in morphology and microglial loss. Each panel represents 5 mice per group.

FIG. 10. Increased apoptosis in 4D4+/CD11b+ microglia was starting at presymptomatic stage and continues during disease progression in EAE mice. C57/B6-EAE mice analyzed in FIGS. 9A-B were analyzed by FACS for apoptotic (AnnexinV+) and necrotic (7AAD+) cells. Each panel represents a pool of 5 mice per group.

FIG. 11. Systemic injection (ip) of anti-6C3 Ab delayed the onset and attenuated severity of EAE-induced mice.

FIGS. 12A-C. Increase of peripheral inflammatory monocytes recruitment leads to indigenous microglia loss in the eye of aged chimera mouse transplanted with bone marrow cells from CX3CR1-GFP 8 weeks-old transgenic mouse. Anatomical distribution of peripheral monocytes in the eye of 24m-old CX3CR1-GFP chimera mouse (A). B and C boxes represent inserts at high magnification. A large number of CX3CR1-GFP peripheral monocytes are present in the vicinity of an almost entirely destroyed part of the retinal ganglion cell layer (A). In the other region (B) of the same retina, the well preserved part of the retinal ganglion cell layer contains a few adjacent CX3CR1-GFP peripheral monocytes. Retinal ganglion cell layer is identified by NeuN.

FIGS. 13A-F. Indigenous microglia loss and increased recruitment of Ly6C+ peripheral inflammatory monocytes in D2 glaucoma mouse. (A) FACS analysis of the retina of 8 weeks-old wt, 8 months-old wt and 8 months-old glaucoma D2 mice. CD11b-gated cells (upper row, boxed area in green) and indigenous microglia (CD11b+/4D4+, lower row). Note, decreased number of CD11b+ cells and CD11b+/4D4+ in both 8 months-old wt and D2 glaucoma mice compared to the young 8 weeks-old wt mice. (B) FACS analysis of the optic nerve of 8 months-old wt and 8 months-old glaucoma D2 mice. CD11b-gated cells (upper row, boxed area in green) and indigenous microglia (CD11b+/4D4+, lower row). Note, decreased number of CD11b+ cells and CD11b+/4D4+ in 8 months-old D2 glaucoma mice compared to the 8 months-old wt mice. (C) FACS analysis of CD11b-gated cells analyzed for 4D4 expression in the retina shows decrease in the number of 4D4+/CD11b+ cells (upper row) and increase in the number of 6C3+/CD11b+ cells (lower row) in the glaucoma D2 mice group. (D) FACS analysis of CD11b-gated cells analyzed for 4D4 expression in the optic nerves shows decrease in the number of 4D4+/CD11b+ cells (upper row) and increase in the number of 6C3+/CD11b+ cells (lower row) in the glaucoma D2 mice group. (E) FACS analysis for apoptosis (upper row) and necrosis (lower row) of retinal indigenous microglia (4D4+) cells shows increase of apoptosis and necrosis in both in 8 months-old wt mice compared to 8 months-old D2 glaucoma mice. (F) Graphic presentation of CD11b+/4D4+ and CD11b+/4D4− cells (upper row) in the retina (left) and optic nerve (right) and of CD11b+/6C3+ and CD11b+/6C3− cells (upper row) in the retina (left) and optic nerve (right).

FIGS. 14A-H. EAE-induced brain derived 6C3+ peripheral monocytes are cytotoxic to the retinal indigenous microglia after intrevitreal transplantation. (A) FACS analysis of the retina of wild type 10 wks-old mice, which had undergone intravitreal transplantation of pretreated with anti-6C3 antibody (left) and iso-type control treated CD11b+/6C3+ brain-derived cells. CD11b-gated cells analysis for 4D4 expression shows a decrease of CD11b+/4D4+ indigenous microglia cells in the iso-type control subgroup (right) compared to the anti-6C3 pretreated subgroup (left)

Note, marked reduction of 4D4+/CD11b+ indigenous microglial (red boxes) and remarkable increase of peripheral monocytes (6C3+/CD11b+) occurred in the iso-type control subgroup (right) compared to subgroup of anti-6C3 pretreated subgroup (left). (B) FACS analysis of retinal indigenous microglia (4D4+ cells) for apoptosis (upper row) and necrosis (lower row) after transplantation of CD11b+/6C3+ brain-derived cells shows marked increase of both apoptosis and necrosis in the iso-type control treated CD11b+/6C3+ subgroup (right) compared to the anti-6C3 pretreated subgroup (left). (C) FACS analysis of the retina of wild type 10 wks-old mice, which had undergone intravitreal transplantation of pretreated with anti-6C3 antibody (left) and iso-type control treated CD11b+/6C3+ spleen-derived cells. CD11b-gated cells analysis for 4D4 expression shows no change of CD11b+/4D4+ indigenous microglia cells in the iso-type control subgroup (right) compared to the anti-6C3 pretreated subgroup (left). Note, a lesser extent of reduction of 4D4+/CD11b+ indigenous microglia (red boxes) and increase of peripheral monocytes (6C3+/CD11b+) occurred in spleen-derived anti-6C3 pretreated subgroup in comparison to the effect of brain-derived anti-6C3 pretreated subgroup. (D) FACS analysis of retinal indigenous microglia (4D4+ cells) for apoptosis (upper row) and necrosis (lower row) after transplantation of CD11b+/6C3+ spleen-derived cells shows no change of both apoptosis and necrosis between the iso-type control and anti-6C3 treated subgroups. (E) Graphic presentation of CD11b+/4D4+ indigenous microglia cells (upper row) and peripheral monocytes (6C3+/CD11b+) (lower row) after transplantation of brain-derived anti-6C3 pretreated (left) and iso-type pretreated (right) CD11b+/6C3+ cells. (F) Graphic presentation of CD11b+/4D4+ indigenous microglia cells (upper row) and peripheral monocytes (6C3+/CD11b+) (lower row) after transplantation of spleen-derived anti-6C3 pretreated (left) and iso-type pretreated (right) CD11b+/6C3+ cells. G. Graphic presentation of retinal indigenous microglia (4D4+ cells) for apoptosis (upper row) and necrosis (lower row) after transplantation of CD11b+/6C3+ brain-derived cells. H. Graphic presentation of retinal indigenous microglia (4D4+ cells) for apoptosis (upper row) and necrosis (lower row) after transplantation of CD11b+/6C3+ spleen-derived cells.

FIGS. 15A-C. Pre-treatment of EAE-induced CNS-derived CD11b+/6C3+ cells with anti-6C3 antibody before the transplantation resulted in preservation of indigenous microglia cells. (A) Confocal images of the retina show a decrease of CD11b+/4D4+ indigenous microglia cells in the iso-type control (right) compared to anti-6C3 pretreated (left) brain-derived CD11b+/6C3+ cells. (B) Confocal images of the retina show no change of CD11b+/4D4+ indigenous microglia cells in the iso-type control (right) compared to anti-6C3 pretreated (left) spleen-derived CD11b+/6C3+ cells. (C) Demonstration of the transplanted CD11b+/6C3+ brain derived cells in the vitreous cavity in a proximity to the retinal ganglion cell layer.

FIG. 16. Deficiency of TGFbeta in the CNS results in widespread microglial loss accompanied by increased recruitment of 6C3+ peripheral monocytes and retinal ganglion cell. Confocal images of retinas from 20d- or 160d-old tg-wt TGFb+/+xIL2TGF-beta and TGFb-deprived CNS TGFb−/−xIL2TGF-beta mice stained for 4D4, NeuN, IBA1 or GFAP, as indicated. Note, no indigenous microglia (4D4+/IBA1+) were identified in 20d-old or in 160d-old mice of TGFb−/−xIL2TGF-beta mice. Moreover, retinal ganglion cells (NeuN) loss (arrows on left) and inner nuclear layer loss (arrows on right) are observed in TGFb−/−xIL2TGF-beta mice at the end-stage only.

FIG. 17 is a pair of bar graphs showing biphasic recruitment of the CD11b+Ly6C+ monocytes to the ischemic brain hemisphere following 1 h MCAO. Total levels of mononuclear cells in the brains of MCAO and control (SHAM) mice are shown in the left panel, and levels of CD11b+Ly6C+ monocytes are shown in the right.

FIG. 18 is a set of three bar graphs showing that “early” CD11b+Ly6C+ monocytes in the ischemic brain display enhanced proliferation and reduced cell death at d3 post MCAO.

FIG. 19 is a set of three bar graphs showing that CD11b+Ly6C+ monocyte frequency in the ischemic brain increased between d7 and d21 despite minimal proliferation.

FIG. 20 is a bar graph showing a biphasic reduction in the number of spleen cells following MCAO.

FIG. 21 is a line graph showing a reduction in the infarct area in animals treated with anti-Ly6C antibody following MCAO (solid line) versus animals treated with an isotype control (dashed line).

FIG. 22 is a set of six bar graphs showing the results of treatment of wild type animals with anti-Ly6C antibody.

FIGS. 23A-D. Activation of the chemotaxis pathway in CD39+ resident microglia in the spinal cord but not in the brain of SOD1 mice. (A) Quantitative nCounter expression profiling of 179 inflammation-related genes was performed in spinal cord-derived CD39+ microglia from SOD1 mice and compared to non-transgenic (Tg) littermates at pre-symptomatic (60d), onset (defined by body-weight loss) and end-stage. Heatmap shows genes with at least 2-fold altered transcription levels. Each row of the heatmap represents an individual gene and each column an individual group in biological triplicates (n=3 arrays for each group of pool of 4-5 mice at each time point). The relative abundance of transcripts is indicated by a color scale (red, high; green, median; blue, low). Bars show relative expression of significantly up or downregulated genes in SOD1 mice from non-Tg littermates at each time-point. 20 significantly upregulated genes are shown in (A) and 38 significantly downregulated genes are shown in (B). (C) Major biological networks activated at disease onset in SOD1 as analyzed by MetaCore™ (GeneGo). Gene expression level has been normalized against geometric mean of six house-keeping genes (CLTC, GAPDH, GUSB, HPRT1, PGK1, TUBBS). (D) Comparative analysis of significantly upregulated genes in CD39+ microglia from spinal cords of SOD1 mice at onset vs. microglia isolated from the brain of the same mice.

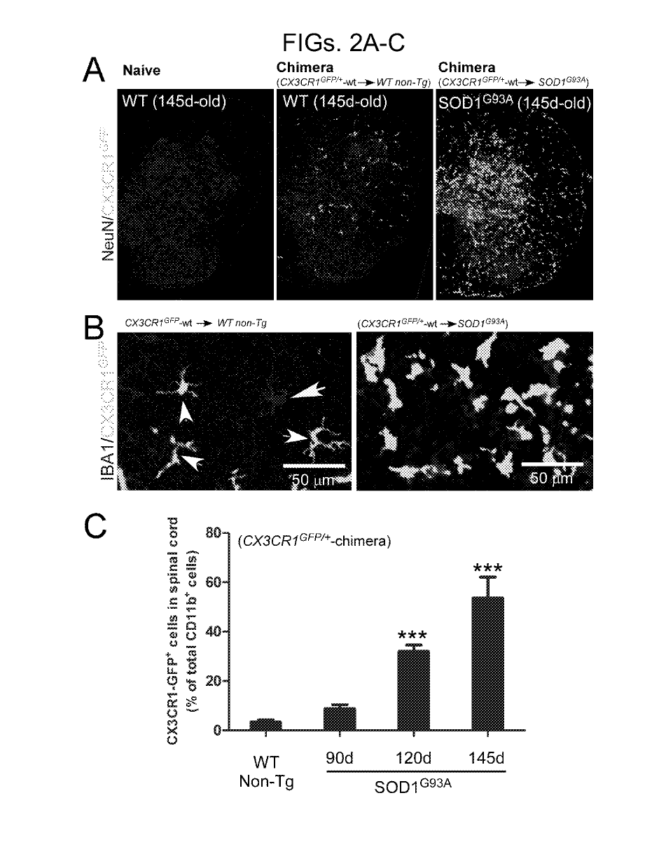

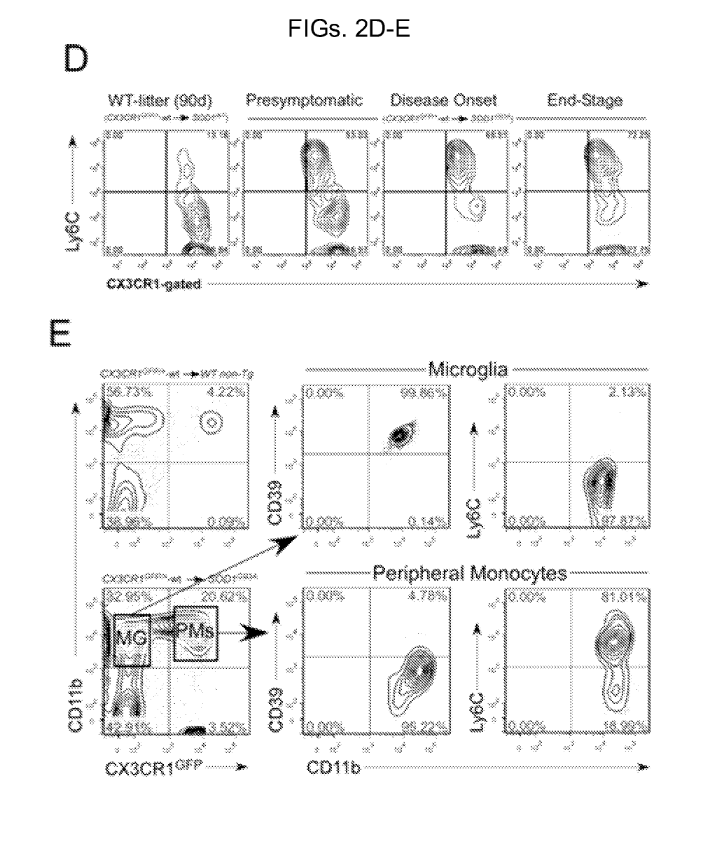

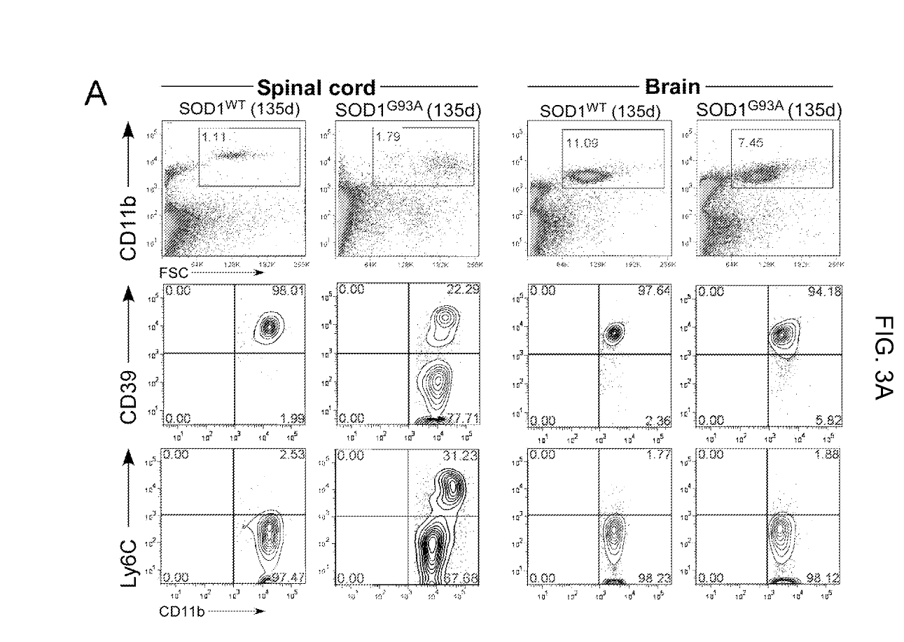

FIGS. 24A-C. Ly6CHi monocytes in the spleen exhibit a pro-inflammatory profile two months prior to clinical disease onset and during disease progression in SOD1 mice. (A) Quantitative nCounter expression profiling of 179 inflammation-related genes showing significantly upregulated and (B) downregulated genes in splenic Ly6CHi monocytes compared to non-Tg littermates of the same mice analyzed in FIG. 2 at pre-symptomatic (30d and 60d of age), disease onset and end-stage. (C) MetaCore™ (GeneGo) analysis showing significantly activated biological networks two months prior to clinical onset.

FIG. 25A-C. mSOD1-microglia induce recruitment of Ly6C+ monocytes. (A) Spinal cord microglia were sorted from donor WT and mSOD1 mice at onset with CD39 mAb and transplanted intracranially into recipient WT or mSOD1 mice at onset. (B) 48 h post-transplantation, myeloid cells were isolated and analyzed by FACS for recruited Ly6C+/CD11b+ monocytes. (C) Quantification of Ly6C+ monocytes in transplanted hemispheres of WT and SOD1 mice.

FIG. 26A-D. Ly6CHi monocytes proliferate and CD39+ microglia undergo apoptosis during disease progression in the spinal cord of SOD1 mice. Spinal cord-isolated myeloid cells at onset (90d), early symptomatic (120d) and late-symptomatic (135d) stages from SOD1 WT and SOD1 mice were analyzed. (A) Microglia viability was evaluated using AnnexinV and 7-AAD for apoptotic and necrotic cells, respectively. Note: no significant apoptosis was detected in Ly6C+ monocytes (not shown). (B) Quantification of microglia viability reveals an approximately 2.5 fold increase in microglial apoptosis at 90d, 120d and 135d in comparison to wild type microglia. (C) Proliferation of CD39+ resident microglia and Ly6C+ monocytes assessed by BrdU incorporation. BrdU was injected (ip) daily for 5 consecutive injections before the spinal cords were analyzed. Wild type mice received the same course of BrdU injection. Spinal cords were excised 5 days after the first BrdU injection. G1-gated CD11b+/CD39+ microglia; G2-gated Ly6CHi and G3-gated Ly6CLow monocytes. (D) Ly6CHi monocytes proliferate 3-4 fold more than Ly6CLow cells during the disease course. Error bars reflect the standard error of multiple measurements with pool of 3-4 mice per group. Statistical analysis between SOD1G93A and non-Tg mice is by t test.

FIGS. 27A-G 4D4+ microglial loss occurs during disease progression in the spinal cord, but not in the brain of SOD1 mice. Representative confocal images show immunohystochemistry of triple staining for 4D4 (microglia), NeuN (neurons) and IBA1 (stains both microglia and peripheral monocytes). (A) Confocal images of whole mount axial sections of spinal cord from wt-litter and SOD1G93A transgenic mice at presymptomatic, disease onset and end-stage, as indicated. Boxed areas represent inserts at high magnification in separate confocal channels. (B) Confocal images of hippocampus areas adjacent to CA1 in wt-litter (top) and SOD1 transgenic mouse (bottom). Representative confocal images of 4 mice per group. (C) Quantification of progressive IBA1+ cell activation and (D) IBA1+ cell area in spinal cords of SOD1G93A relative to wt-litters (dashed line, as fold of induction, %). (E) Quantification analysis of NeuN+ cells and (F) 4D4+ cells in ventral and dorsal horns of spinal cord of SOD1G93A and wt-litter at indicated time points. (n=5-6 per group). Two-way ANOVA, Bonferroni post-tests (G) Western blot analysis of brain and spinal cord of SOD1G93A-mice at indicated stage of the disease (n=3, pool).

FIG. 28. Real-time PCR showed CCL2 expression was significantly upregulated. Relative expression in sALS and fALS against HC were calculated using the comparative Ct (2-ΔΔCt) method. Gene expression level was normalized against geometric mean of three house-keeping genes (GAPDH, TUBB and GRB2). PCRs were run in duplicates per subject.

### DETAILED DESCRIPTION

Microglia serve to protect and preserve neuronal cells from pathogens and facilitate recovery from metabolic insults (Schwartz et al., Trends Neurosci 29:68-74, 2006). In addition, they appear to play a role in the neuropathology of noninfectious inflammatory disorders of the central nervous system, especially those that are autoimmune. Presentation of neural autoantigens to autoreactive T cells by microglia and the attendant secretion of proinflammatory cytokines are thought to facilitate the inflammatory process in diseases such as multiple sclerosis. They also serve as scavengers of damaged myelin following death of oligodendrocytes and the destruction of myelin and may, therefore, promote recovery of myelin damaged by the inflammatory insult (Butovsky et al., J Clin Invest 116:905-915, 2006). In autoimmune diseases such as multiple sclerosis, most data point to a detrimental role of microglia, for example by producing neurotoxic molecules, proinflammatory cytokines, chemokines or by presenting self-antigens (Becher et al., (2000) Glia 29:293-304.; Carson, Glia 40:218-231, 2002; Heppner et al., Nat Med 11:146-152, 2005). Recent studies tried to define distinct roles and anatomical positions for microglia in the pathogenesis of EAE (Greter et al., Nat Med 11:328-334, 2005; Heppner et al., 2005).

As described herein, the monoclonal antibody 6C3, which binds to Ly6C, identifies inflammatory detrimental monocytes associated with CNS pathology; 5E12 (which binds to CD39) and 4D4 mAbs identify resident microglia from infiltrated 6C3+ (Ly6C) monocytes. Using these antibodies it was possible to distinguish between indigenous microglia and infiltrating monocytes participating in neuroinflammatory processes in animal models of MS, AD, ALS, brain stroke and eye-related diseases in mice models such as glaucoma, retinitis pigmentosa and AMD. These recruited peripheral inflammatory monocytes, identified with 6C3, express high levels of IL1beta, IL6, IL17, IL27 and TNFalpha.

In addition, antibodies targeting these inflammatory detrimental monocytes have a therapeutic value. In EAE-, ALS- and stroke-induced (MCAO) mice increased expression and recruitment of 6C3+ (Ly6C+) blood-derived monocytes was associated with the disease progression. Moreover, induced recruitment of 6C3+ monocytes was detected in an animal model of glaucoma that is correlated with retina ganglion cell loss. Passive transfer of recruited 6C3+ inflammatory monocytes from EAE-induced mice to wt-eye significantly induced apoptosis in endogenous microglia. However, pre-treatment of brain-derived CD11b+/6C3+ recruited monocytes with anti-6C3 Ab before the transplantation resulted in preservation of indigenous microglia cells. Interestingly, spleen-derived CD11b+/6C3+ cells does not affect indigenous microglial loss. Systemic injection (ip) of anti-6C3 Ab delayed the onset and attenuated severity of EAE-induced mice Importantly, systemic injection (ip) of anti-6C3 Ab immunomodulates the detrimental phenotype. Thus, anti-6C3 Ab, suppressed IL1beta, IL6, TNF-alpha and induced TGF-beta expression in 6C3+ CNS- and spleen-derived monocytes. Moreover, systemic injection of anti-6C3 Ab in SOD1 mice (ALS mouse model) after disease onset attenuated disease progression and extended survival of SOD1G93A mice at least for 14-18 days. Related to the findings that anti-6C3 treatment induced TGF-beta, a new mouse model was developed that expressed TGF-beta in activated T cells in periphery under control of the IL-2 promoter (IL2TGF-beta). It was hypothesized that if anti-6C3 treatment attenuated disease in the SOD 1 mouse by the induction of TGF-beta in inflammatory monocytes, then TGF-beta may play an important role in the pathologic processes in SOD1 mice. To test this hypothesis the SOD1G93A mice were crossed with IL2TGF-beta-tg mice which provided an endogenous source of TGF-beta; the crossed mice had extended survival as compared to the SOD1 mice of at least 20 days.

In addition, treatment of glaucoma mice with anti-6C3 Ab suppressed the recruitment of 6C3+ monocytes and induced a neuroprotective effect on retinal ganglion cells (RGCs). These demonstrate that antibodies targeting inflammatory detrimental monocytes can be used for the diagnosis and treatment of CNS diseases, autoimmune diseases, inflammatory-associated diseases and diseases of the eye.

There is no Ly6C expression in human monocytes; however, the human equivalent of Ly6CHi monocytes has been described as CD14+/CD16−/CCR2+ monocytes (Geissmann et al., Immunity 19: 71-82 (2003)). There are two functional subsets: short-lived CX3CR1Low/CCR2−/Gr1+ (Ly6CHi) recruited to inflamed tissues in CCR2-dependent manner and a CX3CR1Hi/CCR2−/Gr1− subset (Ly6CLow) characterized by CX3CR1-dependent recruitment to noninflamed tissues.

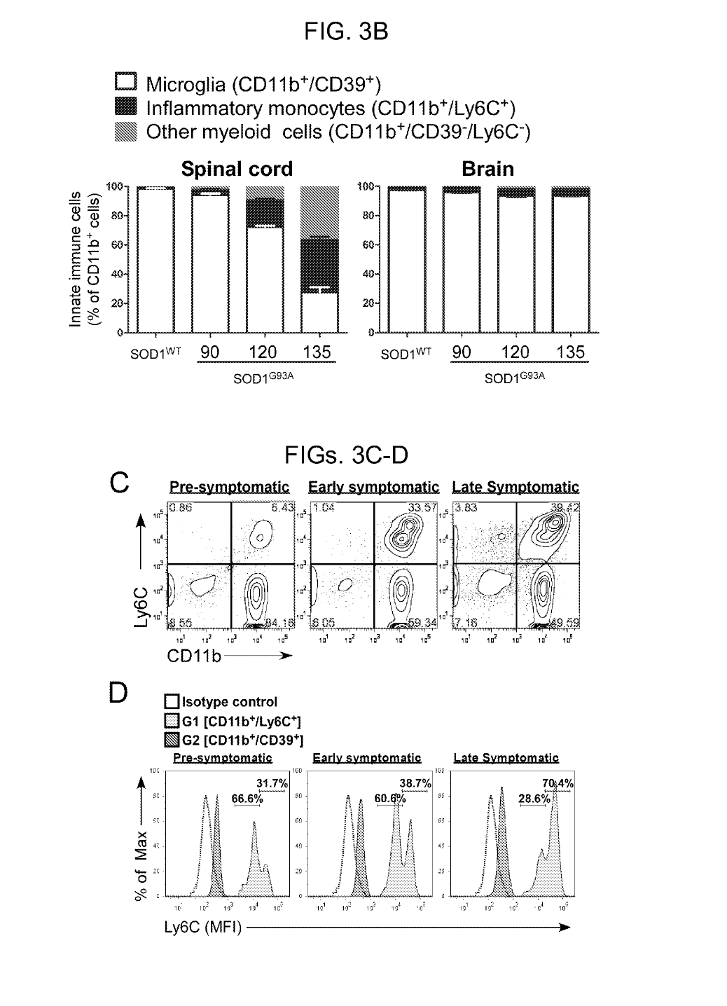

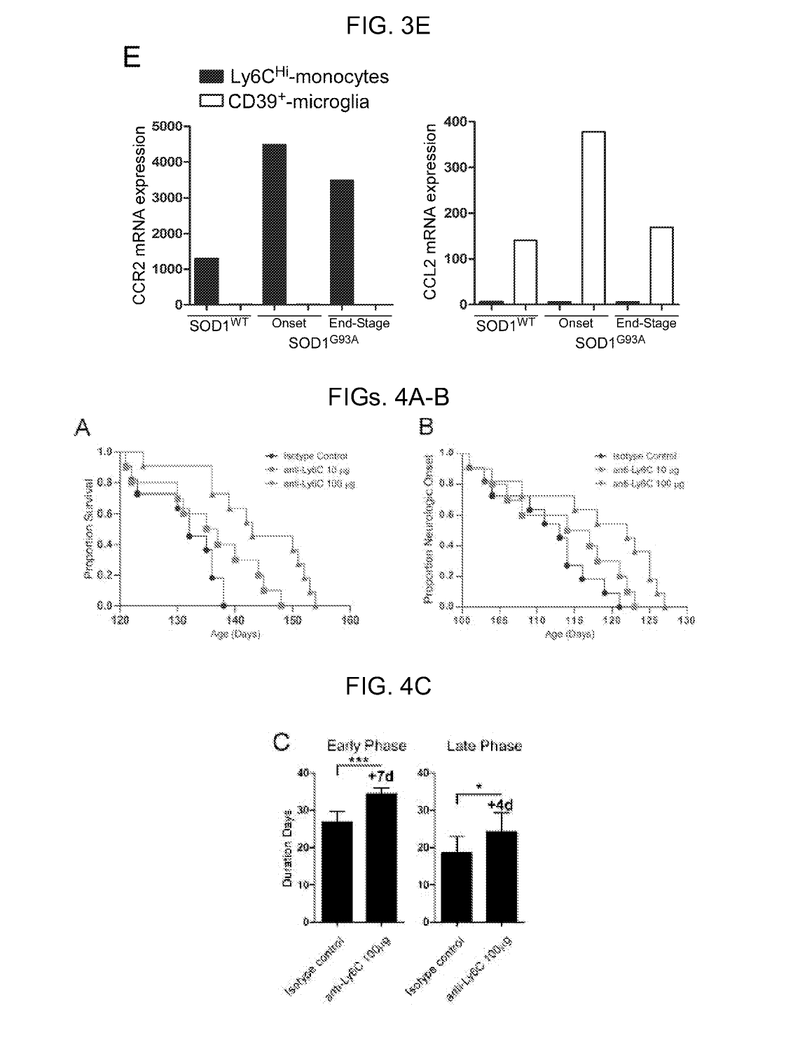

Thus, the functional equivalent target to mouse Ly6C in humans is CCR2. CCL2, also known as monocyte chemoattractant protein-1, the ligand for CCR2, plays a role in various inflammatory diseases (Kang et al., Expert Opin Investig Drugs. 2011 June; 20(6):745-56); in both the mouse model and human ALS, CCL2 is upregulated and is a therapeutic target for treatment of ALS and the other diseases described herein; methods of inhibiting the CCL2-CCR2 axis can be used to block recruitment of CD14+/CD16−/CCR2+ monocytes. CCL2 is upregulated in blood-derived CD14+/CD16−/CCR2+ monocytes in ALS (see Example 18 and FIG. 28). In addition, microglia in SOD1 mice significantly upregulate expression of CCL2 (FIG. 23A and FIG. 3E) and directly mediate recruitment of Ly6C+ monocytes in SOD1 mice (FIG. 25).

Methods of Treatment

The methods described herein can be used for the treatment of certain pathological conditions associated with inflammation, e.g., diseases associated with or caused by the presence of inflammatory monocytes, e.g., Amyotrophic Lateral Sclerosis, stroke, MS, and glaucoma. The methods include administration of a therapeutically effective amount of a compound, e.g., an antibody or small molecule that binds to and inhibits CCL2 or CCR2.

Amyotrophic Lateral Sclerosis (ALS)

ALS is a progressive neurodegenerative disease characterized by injury and cell death of motor neurons which is usually fatal within 2-5 years. Although the majority of cases are sporadic (90%), the most common form of familial ALS is linked to mutations in the Cu/Zn superoxide dismutase 1 (SOD1) gene (Rosen D R. Nature 364: 362 (1993)). In mice, transgenic overexpression of human SOD1 mutant proteins induces a motor neuron disease resembling ALS (Bruijn et al., Neuron 18: 327-338 (1997); Gurney et al., Science 264: 1772-1775 (1994)). There are no treatments for patients with ALS save for supportive care and the drug riluzole which prolongs life by only a few months. Thus, a better understanding of the disease process and development of treatments that can affect the disease course and prolong life represents a critical need for this devastating neurologic disease.

Although ALS is not primarily considered an inflammatory or immune mediated disease, immune mechanisms appear to play a role in the disease. In both patients and animal models of ALS inflammatory responses are observed (McGeer et al., 26: 459-470 (2002)). As described herein, peripheral Ly6C^{Hi} cells play an important role in disease progression in ALS SOD1-Tg mice. It is known that Ly6C^{Hi} monocytes participate in tissue damage and disease pathogenesis in other conditions including EAE (an animal model of MS) (King et al., Blood 113: 3190-3197 (2009)), brain (Dimitrijevic et al., Stroke 38: 1345-1353 (2007)) and heart ischemia (Nahrendorf et al., J Exp Med 204: 3037-3047 (2007)) and atherosclerosis (Combadiere et al., Circulation 117: 1649-1657 (2008)).

The human equivalents of Ly6CHi monocytes (CD14+/CD16− monocytes) (Weber et al., J Leukoc Biol 67: 699-704 (2000)) are well characterized and have been studied in ALS. Henkel et al., reported that there are increased CD14 monocytes in the spinal cord of ALS subjects in close proximity to motor neurons and this was associated with disease progression (Henkel et al., Ann Neurol 55: 221-235 (2004)). Consistent with this, the authors reported increased expression of CCL2 in ALS glial cells. CCL2 is the main ligand for Ly6CHi monocytes. Furthermore, Mantovani et al., reported a decrease of CD14+ cells in the blood of ALS patients and postulated that this related to their early recruitment to CNS areas of primary neurodegeneration (Mantovani et al., J Neuroimmunol 210: 73-9 (2009)).

Glaucoma

Glaucoma is a major cause of preventable blindness making approximately 67 million people throughout the world at risk of blindness (Thylefors et al., Bull World Health Organ 1995; 73(1): 115-21; Quigley, Br J Ophthalmol 1996; 80(5): 389-93). In the United States, more than 2 million people are currently affected and more than 80,000 are legally blind from the disease (Friedman et al., Arch Ophthalmol 2004; 122(4): 532-8). Glaucoma results in a slow, progressive, and selective dysfunction and ultimately apoptotic death of retinal ganglion cells (RGCs), the retinal neurons that project to the brain via the optic nerve (Quigley, Invest Ophthalmol Vis Sci. 2005; 46:2662-70; Guo et al., Invest Ophthalmol Vis Sci. 2005; 46:175-82; Quigley and Addicks, Arch Ophthalmol. 1981; 99:137-43; Quigley, Aust N Z J Ophthalmol. 1995; 23:85-91; Quigley et al., Invest Ophthalmol Vis Sci. 1995; 36:774-86). The precise mechanisms involved in glaucomatous RGC death are not completely understood, but it is widely accepted that pathophysiological events in the retinal ganglion cell layer and at the optic nerve head, through which RGC axons pass, play a prominent role in the development of this neuropathy. Glaucoma progression toward chronic optic nerve atrophy and asynchronous death of retinal ganglion cells has two primary risk factors: age and high intraocular pressure (IOP) (Ahmed et al., Invest Ophthalmol Vis Sci. 2004; 45:1247-1258). However, lowering IOP decelerates, but does not halt, glaucoma, suggesting that therapies targeting the pathogenesis of neurodegeneration might be a more promising approach for intervention. Glaucoma involves gliosis and innate immune responses (see Bosco et al., Invest Ophthalmol Vis Sci. 2008; 49:1437-1446, and references cited therein), suggesting a pathogenic function of local immune cells, consisted of microglia which are believed to be the resident immune surveillance cells in the central nervous system and retina, and peripheral infiltrating monocytes, which are believed to migrate to the area of damage.

It has been shown that in the adult, microglia are quiescent unless pathogens, injury, or stress trigger their proliferation, migration, and activation. Within the healthy adult retina, perivascular and parenchymal resting microglia localize to the inner retina (Langmann, J Leukoc Biol. 2007; 81:1345-1351). It has also been shown that microglia become activated and migratory after RGC axotomy (Thanos, Eur J Neurosci. 1991; 3:1189-1207), ischemia (Chauhan et al., Invest Ophthalmol Vis Sci. 2002; 43 :2969-2976), photoreceptor degeneration (Hughes et al. Invest Ophthalmol Vis Sci. 2003; 44:2229-2234), and endothelin-induced optic neuropathy (Chauhan et al., Invest Ophthalmol Vis Sci. 2004; 45:144-152). In persons with glaucoma, microglia become activated and redistributed within the optic nerve head (ONH) (Neufeld, Arch Ophthalmol. 1999; 117:1050-1056; Tezel et al., Invest Ophthalmol Vis Sci. 2003; 44:3025-3033), producing proinflammatory cytokines, reactive oxygen species, neurotoxic matrix metalloproteinases, and neurotrophic factors. Activated microglia can produce cytokines/chemokines or cytotoxins and have phagocytic activity (Block et al., Nat Rev Neurosci. 2007; 8:57-69; Kim and de Vellis, J Neurosci Res. 2005; 81:302-313), but the specific influence of microglial factors on other retinal cells, including RGCs, is unclear, though potentially linked to glaucoma pathology (Langmann, J Leukoc Biol. 2007; 81:1345-1351; Tezel and Wax, Chem Immunol Allergy. 2007; 92:221-227).

Presently, there are no markers distinguish indigenous micro glia cells from infiltrating hematogenous peripheral monocytes (Shechter et al., PLoS Med 6(7): e1000113 (2009)).

The present inventors have identified two monoclonal antibodies that are unique for adult and primary newborn microglia cells, and an additional clone which specifically identifies peripheral inflammatory monocytes associated with CNS pathology (4D4 and 6C3, respectively). In the glaucoma D2 mouse model, using these new unique biomarkers of indigenous microglia, the earliest pathological event in the development of glaucoma is a decrease in number of indigenous microglia (uniquely stained by 4D4) and an increase in number of peripheral inflammatory macrophages (uniquely stained by 6C3) in the retina and optic nerve.

The anti-6C3 antibody inhibits or modulates infiltrating 6C3 positive peripheral monocytes in retina and optic nerve associated with the disease progression. Modulating these cells could stop retinal ganglion and indigenous microglia cells loss occurring in glaucoma. This has a neuroprotective effect, which may be extended to other types of glaucoma, including primary or secondary, normal tension or primary open angle or angle closure.

Stroke

Ischemic stroke results from transient or permanent reduction in cerebral blood flow. It is one of the main causes of morbidity and mortality worldwide. The mortality from stroke is ˜30%, 80-90% of stroke survivors exhibit motor weakness, and 40-50% experience sensory disturbances (Bogousslaysky et al., 1988. Stroke 19:1083). In the center of the perfusion deficit, cerebral blood flow is typically 80% below normal levels (Hossmann,. A.. 1994. Ann. Neurol. 36:557). Ischemic tissue dies over minutes to many hours (Id.).

Inflammation is also initiated by ischemia at the blood-microvascular endothelial cell interface and contributes significantly to CNS damage. Polymorphonuclear leukocytes rapidly enter injured brain tissue (del Zoppo et al., 2001. Arch. Neurol. 58:669) and white blood cells traverse the blood-brain barrier (BBB) 12-24 h after onset and may provide a source of oxygen-free radicals. Eventually, the infarcted zone is infiltrated with lymphocytes, polymorphonuclear cells, and macrophages (Koroshetz and Moskowitz. 1996. Trends Pharmacol. Sci. 17:227). Neutrophils, important cellular components of the innate immune response, produce a number of potentially harmful substances including toxic oxygen metabolites, destructive enzymes, and proinflammatory cytokines with neurotoxic properties (Li et al., J. Neuroimmunol .116:5 (2001)). Thus, the severity of postischemic injury can be affected by manipulation of the inflammatory response.

Compounds Targeting CCR2 and CCL2

CCL2, also known as monocyte chemoattractant protein-1 (MCP-1), is a chemokine that plays a role in monocyte chemotaxis. The nucleic acid sequence for human CCL2 is available in GenBank at Acc. No. NM_{—}002982.3; the protein sequence can be found at NP_{—}002973.1. See, e.g., Yoshimura and Leonard, Adv. Exp. Med. Biol. 305, 47-56 (1991); and Gronenborn and Clore, Protein Eng. 4 (3), 263-269 (1991). A number of inhibitors of CCR2 are known in the art, including antibodies as well as small molecule inhibitors.

CCR2 is a receptor for CCL2. The receptor mediates agonist-dependent calcium mobilization and inhibition of adenylyl cyclase. Two alternatively spliced transcript variants are expressed by the human CCR2 gene. The first variant (A) encodes a cytoplasmic isoform. It is alternatively spliced in the coding region resulting in a frameshift and use of a downstream stop codon, compared to variant B. Isoform A, genbank accession numbers NM_{—}001123041.2 (nucleic acid) and NP_{—}001116513.2 (amino acid), has a distinct C-terminus and is 14 amino acids longer than isoform B, genbank accession numbers NM_{—}001123396.1 (nucleic acid) and NP_{—}001116868.1 (amino acid); see, e.g., Charo et al., Proc. Natl. Acad. Sci. U.S.A. (1994) 91, 2752-2756. A number of inhibitors of CCR2 are known in the art, including antibodies as well as small molecule inhibitors.

Anti-CCR2 and Anti-CCL2 Antibodies

The methods described herein can include the administration of an antibody that binds to CCR2 or CCL2. The term “antibody” as used herein refers to an immunoglobulin molecule or immunologically active portion thereof, i.e., an antigen-binding portion. Examples of immunologically active portions of immunoglobulin molecules include F(ab) and F(ab′)2 fragments, which retain the ability to bind antigen. Such fragments can be obtained commercially or using methods known in the art. For example F(ab)2 fragments can be generated by treating the antibody with an enzyme such as pepsin, a non-specific endopeptidase that normally produces one F(ab)2 fragment and numerous small peptides of the Fc portion. The resulting F(ab)2 fragment is composed of two disulfide-connected Fab units. The Fc fragment is extensively degraded and can be separated from the F(ab)2 by dialysis, gel filtration or ion exchange chromatography. F(ab) fragments can be generated using papain, a non-specific thiol-endopeptidase that digests IgG molecules, in the presence of a reducing agent, into three fragments of similar size: two Fab fragments and one Fc fragment. When Fc fragments are of interest, papain is the enzyme of choice because it yields a 50,00 Dalton Fc fragment; to isolate the F(ab) fragments, the Fc fragments can be removed, e.g., by affinity purification using protein A/G A number of kits are available commercially for generating F(ab) fragments, including the ImmunoPure IgG1 Fab and F(ab′)₂ Preparation Kit (Pierce Biotechnology, Rockford, Ill.). In addition, commercially available services for generating antigen-binding fragments can be used, e.g., Bio Express, West Lebanon, N.H.

The antibody can be a polyclonal, monoclonal, recombinant, e.g., a chimeric, de-immunized or humanized, fully human, non-human, e.g., murine, or single chain antibody. In some embodiments the antibody has effector function and can fix complement. In some embodiments, the antibody has reduced or no ability to bind an Fc receptor. For example, the antibody can be an isotype or subtype, fragment or other mutant, which does not support binding to an Fc receptor, e.g., it has a mutagenized or deleted Fc receptor binding region.

Methods for making monoclonal antibodies are known in the art. See, e.g., Harlow, et. al., editors, Antibodies: A Laboratory Manual (1988).

In addition to utilizing whole antibodies, the methods described herein can include the use of binding portions of such antibodies. Such binding portions include Fab fragments, F(ab′)₂ fragments, and Fv fragments. These antibody fragments can be made by conventional procedures, such as proteolytic fragmentation procedures, as described in J. Goding, Monoclonal Antibodies: Principles and Practice, pp. 98-118 (N.Y. Academic Press 1983).

Chimeric, humanized, de-immunized, or completely human antibodies are desirable for applications which include repeated administration, e.g., therapeutic treatment of human subjects.

Chimeric antibodies generally contain portions of two different antibodies, typically of two different species. Generally, such antibodies contain human constant regions and variable regions from another species, e.g., murine variable regions. For example, mouse/human chimeric antibodies have been reported which exhibit binding characteristics of the parental mouse antibody, and effector functions associated with the human constant region. See, e.g., Cabilly et al., U.S. Pat. No. 4,816,567; Shoemaker et al., U.S. Pat. No. 4,978,745; Beavers et al., U.S. Pat. No. 4,975,369; and Boss et al., U.S. Pat. No. 4,816,397, all of which are incorporated by reference herein. Generally, these chimeric antibodies are constructed by preparing a genomic gene library from DNA extracted from pre-existing murine hybridomas (Nishimura et al., Cancer Research, 47:999 (1987)). The library is then screened for variable region genes from both heavy and light chains exhibiting the correct antibody fragment rearrangement patterns. Alternatively, cDNA libraries are prepared from RNA extracted from the hybridomas and screened, or the variable regions are obtained by polymerase chain reaction. The cloned variable region genes are then ligated into an expression vector containing cloned cassettes of the appropriate heavy or light chain human constant region gene. The chimeric genes can then be expressed in a cell line of choice, e.g., a murine myeloma line. Such chimeric antibodies have been used in human therapy.

Humanized antibodies are known in the art. Typically, “humanization” results in an antibody that is less immunogenic, with complete retention of the antigen-binding properties of the original molecule. In order to retain all the antigen-binding properties of the original antibody, the structure of its combining-site has to be faithfully reproduced in the “humanized” version. This can potentially be achieved by transplanting the combining site of the nonhuman antibody onto a human framework, either (a) by grafting the entire nonhuman variable domains onto human constant regions to generate a chimeric antibody (Morrison et al., Proc. Natl. Acad. Sci., USA 81:6801 (1984); Morrison and Oi, Adv. Immunol. 44:65 (1988) (which preserves the ligand-binding properties, but which also retains the immunogenicity of the nonhuman variable domains); (b) by grafting only the nonhuman CDRs onto human framework and constant regions with or without retention of critical framework residues (Jones et al. Nature, 321:522 (1986); Verhoeyen et al., Science 239:1539 (1988)); or (c) by transplanting the entire nonhuman variable domains (to preserve ligand-binding properties) but also “cloaking” them with a human-like surface through judicious replacement of exposed residues (to reduce antigenicity) (Padlan, Molec. Immunol. 28:489 (1991)).

Humanization by CDR grafting typically involves transplanting only the CDRs onto human fragment onto human framework and constant regions. Theoretically, this should substantially eliminate immunogenicity (except if allotypic or idiotypic differences exist). However, it has been reported that some framework residues of the original antibody also need to be preserved (Riechmann et al., Nature 332:323 (1988); Queen et al., Proc. Natl. Acad. Sci. USA 86:10,029 (1989)). The framework residues which need to be preserved can be identified by computer modeling. Alternatively, critical framework residues may potentially be identified by comparing known antibody combining site structures (Padlan, Molec. Immun 31(3):169-217 (1994)). The invention also includes partially humanized antibodies, in which the 6 CDRs of the heavy and light chains and a limited number of structural amino acids of the murine monoclonal antibody are grafted by recombinant technology to the CDR-depleted human IgG scaffold (Jones et al., Nature 321:522-525 (1986)).

Deimmunized antibodies are made by replacing immunogenic epitopes in the murine variable domains with benign amino acid sequences, resulting in a deimmunized variable domain. The deimmunized variable domains are linked genetically to human IgG constant domains to yield a deimmunized antibody (Biovation, Aberdeen, Scotland).

The antibody can also be a single chain antibody. A single-chain antibody (scFV) can be engineered (see, for example, Colcher et al., Ann. N.Y. Acad. Sci. 880:263-80 (1999); and Reiter, Clin. Cancer Res. 2:245-52 (1996)). The single chain antibody can be dimerized or multimerized to generate multivalent antibodies having specificities for different epitopes of the same target protein. In some embodiments, the antibody is monovalent, e.g., as described in Abbs et al., Ther. Immunol. 1(6):325-31 (1994), incorporated herein by reference.

A number of anti-CCR2 and anti-CCL2 antibodies are known in the art, including those described in U.S. Pat. Nos. 6,312,689, 6,084,075, 6,406,694, 6,406,865, 6,696,550, 6,727,349, 7,442,775, and/or 7,858,318; or in US Pre-Grant Publication No. 20110059107. In some embodiments the antibodies are human, humanized or chimeric, see, e.g., U.S. Pat. Nos. 6,696,550, 5,859,205, 5,693,762, 6,075,181, and US Pre-Grant Publication No. 20070111259. In some embodiments, the antibody is an inhibitory or blocking antibody, e.g., a human CCR2 blocking antibody such as MLN1202 (Millennium Pharmaceuticals, Cambridge, Mass.), or a human antibody that neutralizes human CCL2, e.g., carlumab (CNTO 888; Centocor, Inc.); see Loberg et al., Cancer. Res. 67(19):9417 (2007).

Anti-CCR2 antibodies are available commercially from AbD Serotec; ABR, now sold as Thermo Scientific Pierce Antibodies; Acris Antibodies GmbH; antibodies-online; Aviva Systems Biology; BioLegend; Biorbyt; Bioss Inc.; BioVision; Creative Biomart; eBioscience; EMD Millipore; Fitzgerald Industries International; GeneTex; GenWay Biotech, Inc.; IMGENEX; IMMUNOSTEP S.L; Invitrogen; LifeSpan BioSciences; MyBioSource.com; Novus Biologicals; OriGene Technologies; ProSci, Inc; Raybiotech, Inc.; Rockland Immunochemicals, Inc.; Shenandoah Biotechnology; Sigma-Aldrich; and United States Biological.

Anti-CCL2 antibodies are available commercially from 3H Biomedical AB; Abcam; AbD Serotec; Abgent; Abnova Corporation; ABR, now sold as Thermo Scientific Pierce Antibodies; Acris Antibodies GmbH; Advanced Targeting Systems; Antigenix America Inc.; ARP American Research Products, Inc.; Atlas Antibodies; Aviva Systems Biology; BD Biosciences; Bethyl Laboratories; BioLegend; BioVision; CEDARLANE Laboratories Limited; Cell Sciences; Cell Signaling Technology; Creative Biomart; eBioscience; EMD Millipore; Fitzgerald Industries International; GeneTex; GenWay Biotech, Inc.; Hycult Biotech; Invitrogen; LifeSpan BioSciences; MBL International; Novus Biologicals; OriGene Technologies; PeproTech; ProSci, Inc.; R&D Systems; Randox Life Sciences; Raybiotech, Inc.; Rockland Immunochemicals, Inc.; Santa Cruz Biotechnology, Inc.; and Sigma-Aldrich.

Small Molecule Inhibitors of CCR2

A large number of CCR2 antagonists and inhibitors are known in the art; see, e.g., US Pre-Grant Publication Nos. 20090112004 (phenylamino substituted quaternary salt compounds); 20090048238 (biaryl derivatives); 20090029963 (pyrazol derivatives); 20090023713; 20090012063 (imidazole derivatives); 20080176883 (aminopyrrolidines); 20080081803 (heterocyclic cyclopentyl tetrahydroisoquinolines and tetrahydropyridopyridines); 20100056509 (heteroaryl sulfonamides); 20100152186 (triazolyl pyridyl benzenesulfonamides); 20060074121 (bicyclic and bridged nitrogen heterocycles); WO/2009/009740 (fused heteroaryl pyridyl and phenyl benzenesuflonamides); and WO04/050024; specific inhibitors include N-((1R,3S)-3-isopropyl-3-{[3-(trifluoromethyl)-7,8-dihydro-1,6-naph-thyridin-6(5H)-yl]carbonyl}cyclopentyl)-N-[(3S,4S)-3-methoxytetrahydro-2H-pyran-4-yl]amine; 3[(3S,4R)-1-((1R,3S)-3-isopropyl-2-oxo-3-{[6-(trifluoromethyl)-2H-1,3-benz-oxazin-3(4H)-yl]methyl}cyclopentyl)-3-methylpiperidin-4-yl]benzoic acid; (3S,48)-N-((1R,3S)-3-isopropyl-3-{[7-(trifluoromethyl)-3,4-dihydroisoquinolin-2(1B)-yl]carbonyl}cyclopentyl)-3-methyltetrahydro-2H-p-yran-4-aminium; 3-[(3S,4R or 3R,4S)-1-((1R,3S)-3-Isopropyl-3-{[6-(trifluoromethyl)-2H-1,3-benzoxazin-3-(4H)-yl]carbonyl}cyclopentyl)-3-methylpiperidin-4-yl]benzoic acid; INCB3284; Eotaxin-3; PF-04178903(Pfizer) and pharmaceutically acceptable salts thereof.

CCL2 antagonists and inhibitors are also known in the art, e.g., bindarit (2-((1-benzyl-1H-indazol-3-yl)methoxy)-2-methylpropionic acid); AZD2423 (AstraZeneca); NOX-E36 (40-nucleotide L-RNA oligonucleotide linked to 40 kDa PEG; NOXXON Pharma AG); dominant negative peptides and nucleic acids encoding those peptides (see, e.g., Kiyota et al., Mol Ther. 17(5): 803-809 (2009), and 20070004906); and those described in U.S. Pat. Nos. 7,297,696; 6,962,926; 6,737,435 (indole derivatives); U.S. Pat. No. 6,569,888 (indole derivatives); U.S. Pat. Nos. 6,441,004; 6,479,527 (Bicyclic pyrrole derivatives); US Pre-Grant Publication Nos. 20050054668; 20050026975; 20040198719; 20040047860; see also Howard and Yoshimura, Expert Opinion on Therapeutic Patents, 11(7):1147-1151 (2001).

Methods of Administration

The methods described herein include the use of pharmaceutical compositions, which include compounds that target CCR2 or CCL2 as active ingredients.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions, e.g., anti-inflammatory drugs as are known in the art.

Pharmaceutical compositions are typically formulated to be compatible with its intended route of administration. Examples of routes of administration include parenteral, e.g., intravenous, intradermal, subcutaneous, oral, nasal, transdermal (topical), transmucosal, and rectal administration. The route of administration can be selected by one of skill on the art and will depend on the nature of the active compound.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, N.Y.). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical compositions can also be prepared in the form of suppositories (e.g., with conventional suppository bases such as cocoa butter and other glycerides) or retention enemas for rectal delivery.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

### EXAMPLES

The invention is further described in the following examples, which do not limit the scope of the invention described in the claims.

### Example 1

### Reciprocal Expression of Ly6C and CD39 in Inflammatory Monocytes vs. CNS-Resident Microglia in WT and SOD1 Mice

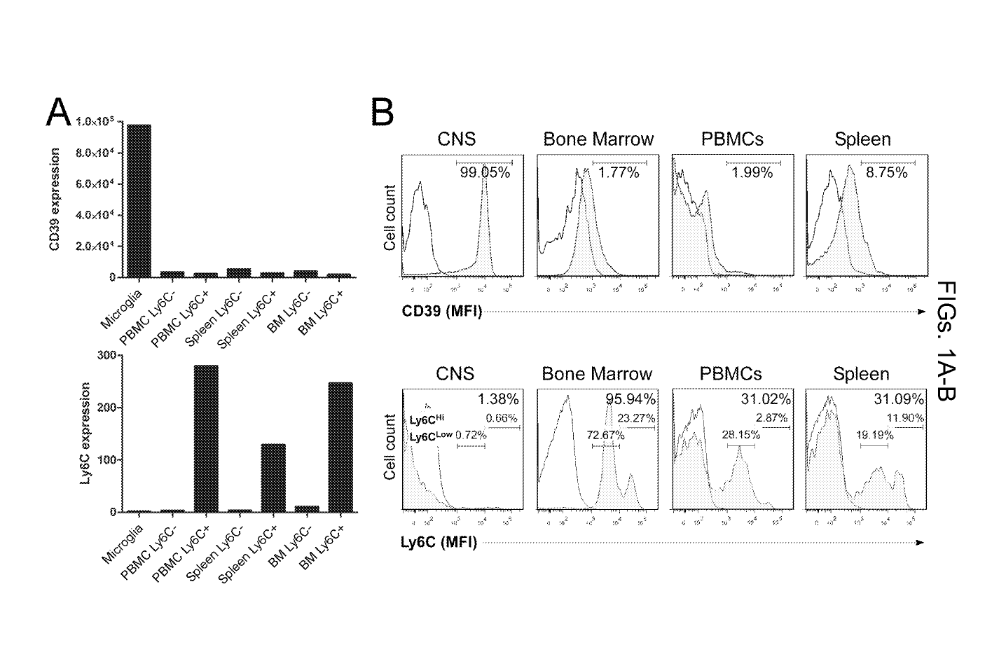

One of the issues confronting the study of monocytes/macrophages and their role in CNS inflammation in neurodegenerative diseases such as ALS is the ability to distinguish infiltrating peripheral monocytes from indigenous, resident microglia in the CNS. During the course of an investigation of immune markers for monocytes and microglia, it was discovered that Ly6C and CD39 (an ectonucleotidase expressed on a subset of Tregs (Gandhi et al., Nat Immunol 11: 846-853 (2010); Fletcher et al., J Immunol 183: 7602-7610 (2009); Borsellino et al., Blood 110: 1225-1232 (2007)) and on microglia in naïve brain (Braun et al., Eur J Neurosci 12: 4357-4366 (2000))) distinguish non overlapping populations of peripheral monocytes and indigenous microglia (FIG. 1). Microglia were isolated from naive adult brains (perfused to remove non-CNS cells) and CD39 and Ly6C expression were compared on microglia vs. Ly6C+and Ly6C-monocytes isolated from PBMC, spleen and bone marrow. As shown by real-time quantitative RT-PCR (qRT-PCR) and FACS, CD39 and Ly6C identify reciprocal populations.

To investigate whether CD39 and Ly6C are also reciprocally expressed in ALS chimeras were generated in which donor peripheral monocytes expressing GFP under the CX3CR1 promoter (Jung et al., Mol Cell Biol 20: 4106-4114 (2000)) were transplanted into recipient irradiated SOD1G93A or non-Tg WT mice. In these chimeras, peripheral monocytes are easily distinguished from microglia by FACS. There was progressive recruitment of CX3CR1-GFP+ monocytes in SOD1G93A mice (FIG. 2A-D) which were Ly6C+ and CD39 negative. (FIG. 2E). Thus, CD39 and Ly6C are specific markers for these reciprocal populations and allow the investigation of characteristics of recruited Ly6C+ cells and contrast them with CNS-resident microglia in SOD1 mice. Of note, Ly6CHi recruited monocytes express low levels of CX3CR1 during all disease stages, resembling the Ly6CHi/CX3CR1Low pro-inflammatory phenotype (Geissmann et al., Immunity 19: 71-82 (2003)). Irradiation sensitizes animals to CNS infiltration by monocytes (Mildner et al., Nat Neurosci 10: 1544-1553 (2007)). These chimeric experiments demonstrate a reciprocal relationship between CD39 and Ly6C.

### Example 2

### Ly6CHi Monocytes Infiltrate the Spinal Cord and CD39+ Microglia Upregulate CCL2 with Disease Progression in SOD1G93A Mice

To investigate the CCR2-CCL2 axis, the presence of CD11b+/Ly6C+ and CD11b+/CD39+ cells in CNS of SOD1 mice was evaluated during disease progression.

SOD1^{G93A} and WT mice were transplanted with BM cells from CX3CR1-GFP^{+/-} Spinal cords were taken at age of 145d (end-stage). As shown in FIG. 3A, in the spinal cords of wt mice, 98% of CD11b+ cells were CD39+ and 2% were Ly6C+. At end stage of disease (135 days) 31% of CD11b cells were Ly6C+. No change in Ly6C+ cells occurred in the brain with disease (FIG. 3A) indicating a relationship between the recruitment of Ly6C+ cells and the areas damaged in the CNS of SOD1 mice. The percentage of CD11b+/Ly6C+ and CD11b+/CD39+ cells in the CNS was quantified over time and found an increase which began during the pre-symptomatic phase and which increased as the disease progressed (FIG. 3B,C). As shown in FIG. 3D, Ly6C expression was upregulated with disease progression and CD39+ microglia remain negative for Ly6C. CCL2 is required for the recruitment of Ly6CHi monocytes to areas of inflammation (Mildner et al., Nat Neurosci 10: 1544-1553 (2007); Qu et al., J Exp Med 200: 1231-1241 (2004); Mildner et al., Brain 132: 2487-2500 (2009); Osterholzer et al., J Immunol 183: 8044-8053 (2009)). CCL2 interacts with CCR2 receptors on the surface of Ly6CHi monocytes. Thus it was examined whether CCR2 was upregulated on Ly6CHi monocytes in the spleen and whether CCL2 was upregulated on CD39+ microglia in CNS during the course of disease in SOD1 mice. As shown in FIG. 3E, CCR2 was upregulated on splenic Ly6CHi monocytes both at disease onset and end stage of disease. This was paralleled by an upregulation of CCL2 on CD39+ microglia at disease onset. There was a reciprocal relationship between these cell types as Ly6CHi monocytes do not express CCL2 and CD39+ microglia do not express CCR2. Thus, expression of CCL2 on microglia plays a role in the recruitment of Ly6CHi monocytes to the CNS. These data also demonstrated changes in the peripheral immune system at early stages of the disease. Of note, CCL2 expression on CD39+ microglia decreased at end-stage disease.

### Example 3

### Anti-Ly6C mAb Treatment Delays Disease Onset and Extends Survival in SOD1G93A Mice

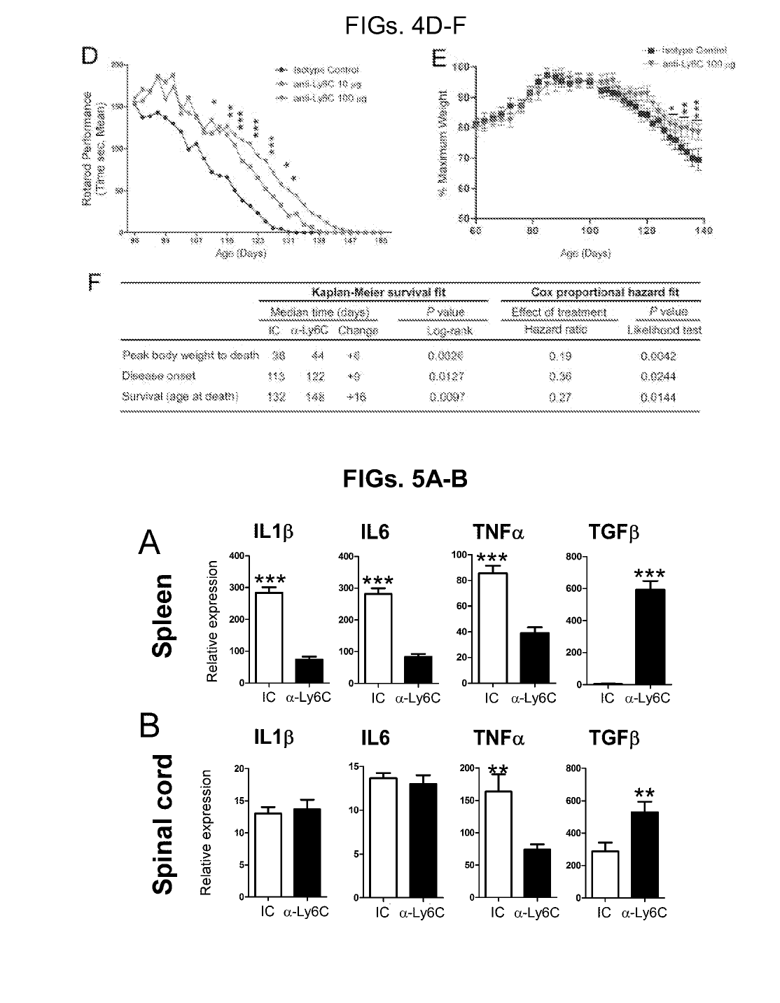

Based on the finding that Ly6CHi monocytes infiltrate the spinal cord and appear to participate in disease progression, SOD1G93A mice were treated with anti-Ly6C mAb to determine if modulation of Ly6CHi monocytes affected disease progression. Animals were treated (i.p.) each second day beginning at disease onset (defined by body weight loss) until end-stage disease. Body weight (daily), clinical neurologic score (daily) and rotarod performance (3×/week) were monitored. As shown in FIG. 4, treatment with 100 ug anti-Ly6C antibody prolonged survival by 16 days (P=0.0097), extended time to reach a neurologic score (curling of toes and dragging of one limb (33)) of two by 9 days (P=0.0127), delayed early (P<0.001) and late (P<0.05) disease onset, enhanced rotarod performance and reduced weight loss (P=0.001 at day 137). Results are representative of three independent experiments in female animals. In the other two experiments (n=7-9/group) treatment with 100ug anti-LyC6 mAb extended survival by 18 days and 8 days. No significant effects were observed in animals treated with 10 ug or 1 ug dosages.

### Example 4

### Anti-Ly6C mAb Treatment Affects the Cytokine Profile of Ly6C^{Hi} Monocytes in the Spleen and Spinal Cord of SOD1^{G93A} Mice

To examine the effects of anti-Ly6C treatment, CD11b⁺/Ly6C^{Hi} cells were sorted from the spinal cord and the spleen from control and anti-Ly6C-treated animals after one month of treatment (age 120 days). As shown in FIG. 5, in the spleen, anti-Ly6C mAb suppressed IL-1β, IL-6 and TNF-α, and increased TGF-beta. In the spinal cord, there were no changes in IL-1β or IL-6, and similar effects as in the spleen were observed for TNF-α and TGF-beta. Thus systemic treatment with anti-Ly6C antibody modulated Ly6C^{Hi} monocytes towards a less pro-inflammatory phenotype in both the periphery and spinal cord.

### Example 5

### Anti-Ly6C mAb Treatment Decreases Infiltration of Ly6C (CD169) Monocytes Into the Spinal Cord and Attenuates Neuronal Loss

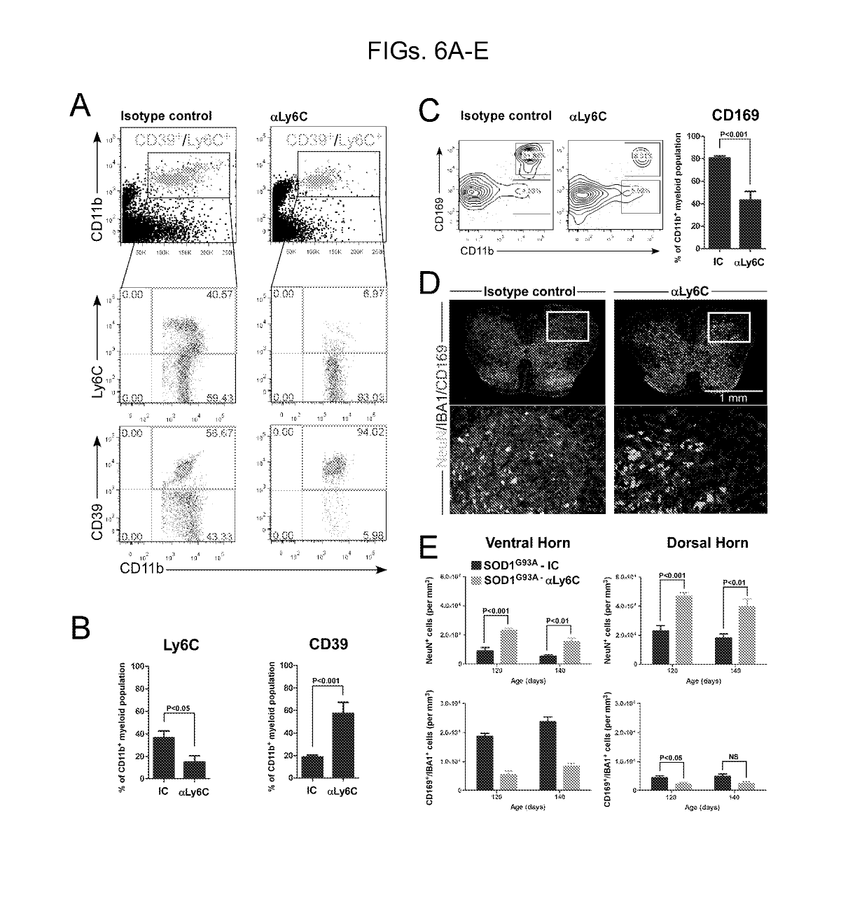

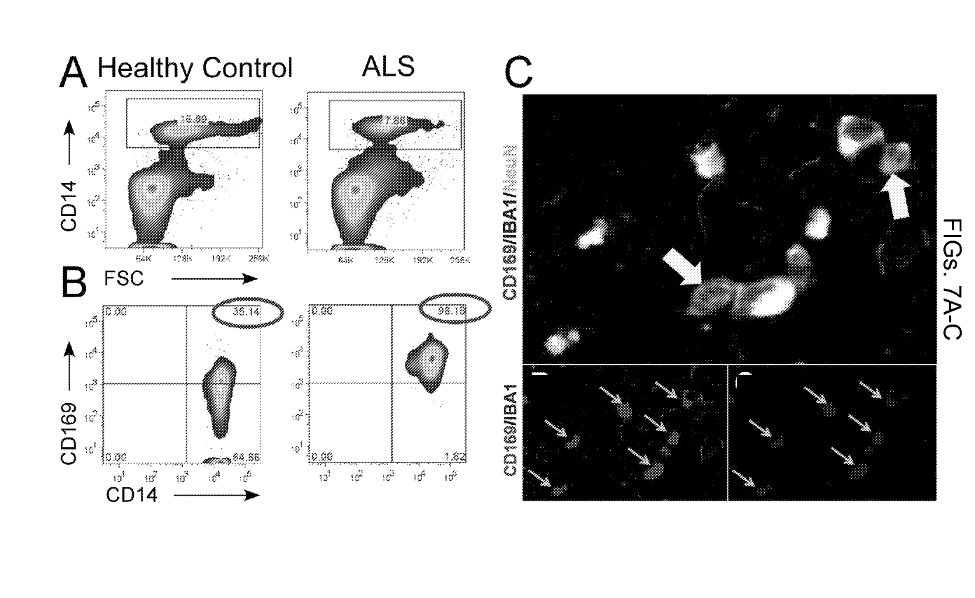

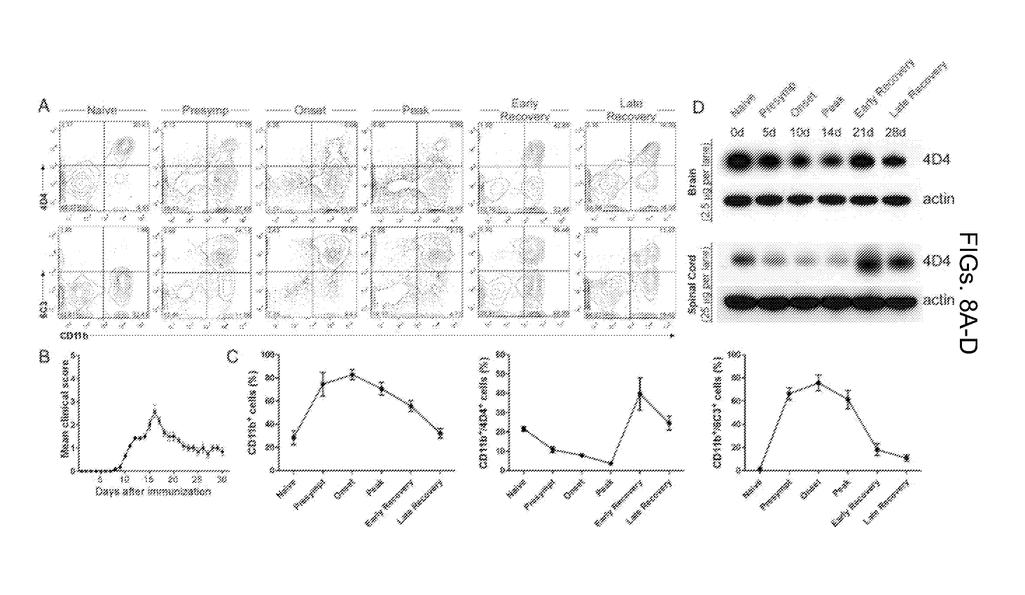

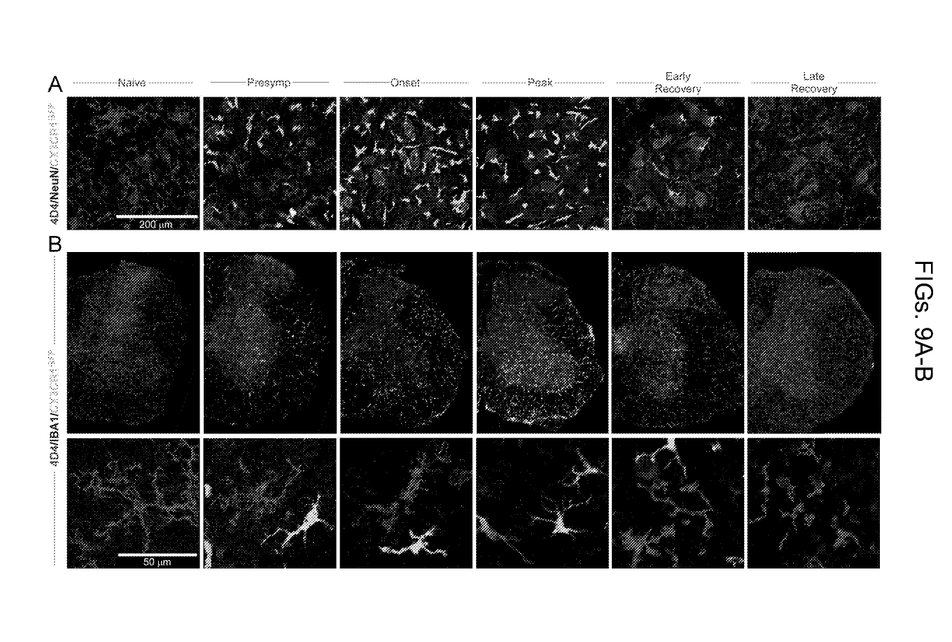

To further investigate the effect of anti-Ly6C mAb treatment in the SOD1 mouse model, FACS analysis was performed to determine if treatment affected the infiltration of Ly6C^{Hi} monocytes to the spinal cord. At 30 days after treatment, there was a marked decrease in the percentage of Ly6C⁺ cells as shown both by FACS (FIG. 6A) and quantitative analysis (FIG. 6B). Also as shown (FIGS. 6A, B) there was a concomitant increase in CD39⁺ microglial cells. Because these experiments included both treatment with anti-Ly6C antibody and use of anti-Ly6C antibody to measure Ly6C in the spinal cord, it is possible that the decrease observed could have been an artifact related to the antibody and not to actual loss of cells. To address this, CD169 mAb was used, which was co-expressed on Ly6C^{Hi} inflammatory monocytes and which, like Ly6C, has a reciprocal relationship with CD39⁺ microglia. As shown in FIG. 6C, there was a similar decrease in CD169⁺ monocytes in the spinal cord of SOD1 mice following anti-Ly6C treatment by FACS analysis. It was then asked whether anti-Ly6C treatment affected neurons in the spinal cord of SOD1 mouse. As shown in FIGS. 6D and E, there was an increase in the numbers of neurons both in the dorsal and ventral horns of anti-Ly6C treated animals. Concomitant with this, there was a decrease in the number of inflammatory monocytes (CD169⁺ cells). These results demonstrated that peripheral Ly6C^{Hi} cells play an important role in disease progression

### Example 6

### CD169 is Upregulated on Inflammatory Monocytes in Blood and Found in the Ventral Horn of Spinal Cord in ALS

To test for inflammatory monocytes, blood samples of ALS patients were analyzed for expression of CD169 in blood CD14⁺ monocytes using FACS. The percentage of CD14⁺ cells was decreased in ALS patients and the expression of CD169 on peripheral inflammatory monocytes was increased as compared to normal healthy subjects (FIG. 7AB). Mantovani et al., also reported a decrease of CD14⁺ cells in the blood of ALS patients and postulated that the decrease was related to recruitment to CNS areas of primary neurodegeneration (Mantovani et al., J Neuroimmunol 210: 73-79 (2009)). Spinal cord cross-sections from ALS patients were analyzed at the end stage of the disease; numerous CD169⁺ peripheral monocytes infiltrated the spinal cord in close proximity to degenerative NeuN⁺ neurons in ventral horn (FIG. 7C). No CD169⁺ cells were found in parenchyma of normal spinal cord specimens.

### Example 7

### Anti-Ly6C (6C3) mAb Treatment Attenuates Clinical Symptoms, Delays Disease Onset and Attenuates Severity in a Mouse Model of MS

To identify unique biomarkers for indigenous microglia and peripheral inflammatory monocytes, a high throughout screen was designed to identify unique hybridoma antibodies against peripheral bone marrow (BM)-derived monocytes and adult microglia cells.

Adult Lewis rats were vaccinated (ip) five times (two weeks apart) with adult freshly isolated microglia cells (5-10×106 per vaccination) sorted with CD45-PerCp and CD11b-PeCy7 antibodies (BD Biosciences) from brains and spinal cords of C57/B16J (8-10 weeks old) mice. The first vaccination was performed with Complete Freund's Adjuvant and then next 3 injections with Incomplete Freund's Adjuvant with live microglia. The final boost was with cells injected both i.v. and i.p. to generate rat hybridoma cells producing specific microglia antibodies. Hybridoma positive oligoclones for microglia or peripheral monocytes were screened and identified by FACS for microglia positive/BM-monocytes negative antibody using pooled murine CD11b⁺/CD45^{Low} adult microglia isolated from C57/B16J mice and GFP+ bone marrow (BM)-derived monocytes isolated from CX3CR1-GFP mice. 3-5 additional subcloning steps were performed to generate monoclones with desired immunoreactivity to adult indigenous microglia or peripheral BM derived monocytes, which produced the 4D4, 6C3 and 5E12 monoclonal antibodies (mAbs). Hybridoma cells producing 4D4, CD39 (5E12) and Ly6C (6C3) mAb were grown in bioreactor (Integra) to produce sufficient amount of antibodies for systemic injections in SOD1 mice.

The 4D4 and 6C3 antibodies distinguish between indigenous microglia and infiltrating monocytes participating in neuroinflammatory processes in animal models of multiple sclerosis (EAE). In contrast to conventional interpretation of the detrimental role of microgliosis, these unique microglia biomarkers revealed that the indigenous 4D4⁺ microglia undergo apoptosis and decrease during disease progression in EAE mice (FIG. 8).

Both an increased expression of Ly6C on splenic monocytes and recruitment of the Ly6CHi monocyte subset into the CNS of EAE mice were associated with the disease progression. In order to characterize infiltrating peripheral monocytes in EAE mice and to evaluate the specificity of 4D4 and 6C3 mAbs to indigenous microglia and recruited monocytes, EAE-chimera mice were generated transplanted with wt BM-derived cells from tg mice expressing GFP under CX3CR1 (all myeloid cells, including monocytes). There was increased recruitment and expression of Ly6C on BM-derived GFP+ recruited monocytes during disease progression. In addition, GFP+ recruited BM-derived monocytes did not express the 4D4 marker for indigenous microglia and were positive for Ly6C (FIGS. 9A-B).

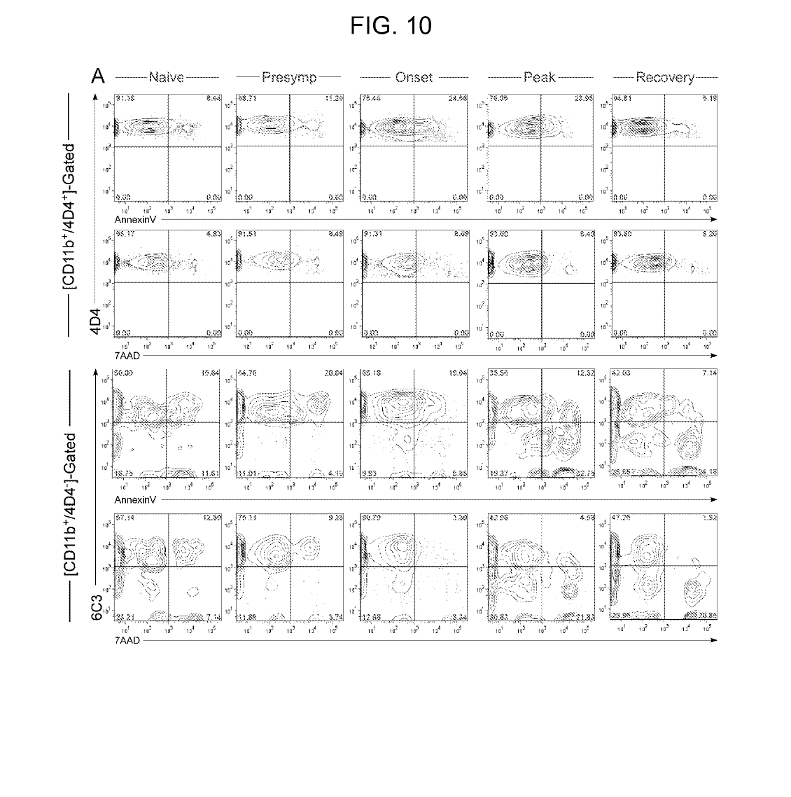

Consistent with the data presented in FIG. 9, there was increased apoptosis and loss of indigenous 4D4+ microglia in the spinal cord of EAE mice (FIG. 10).

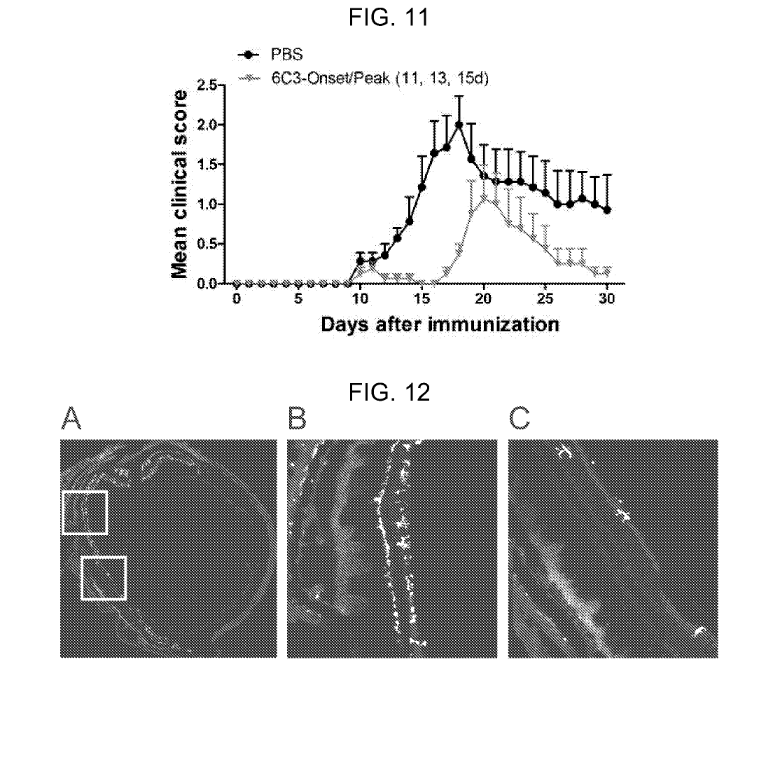

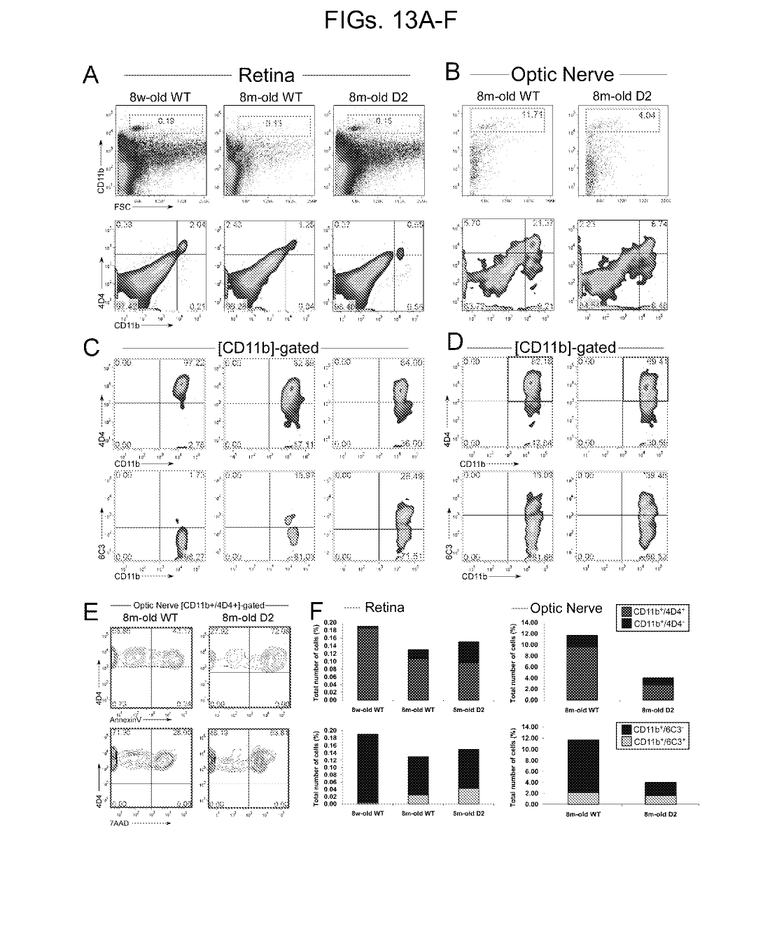

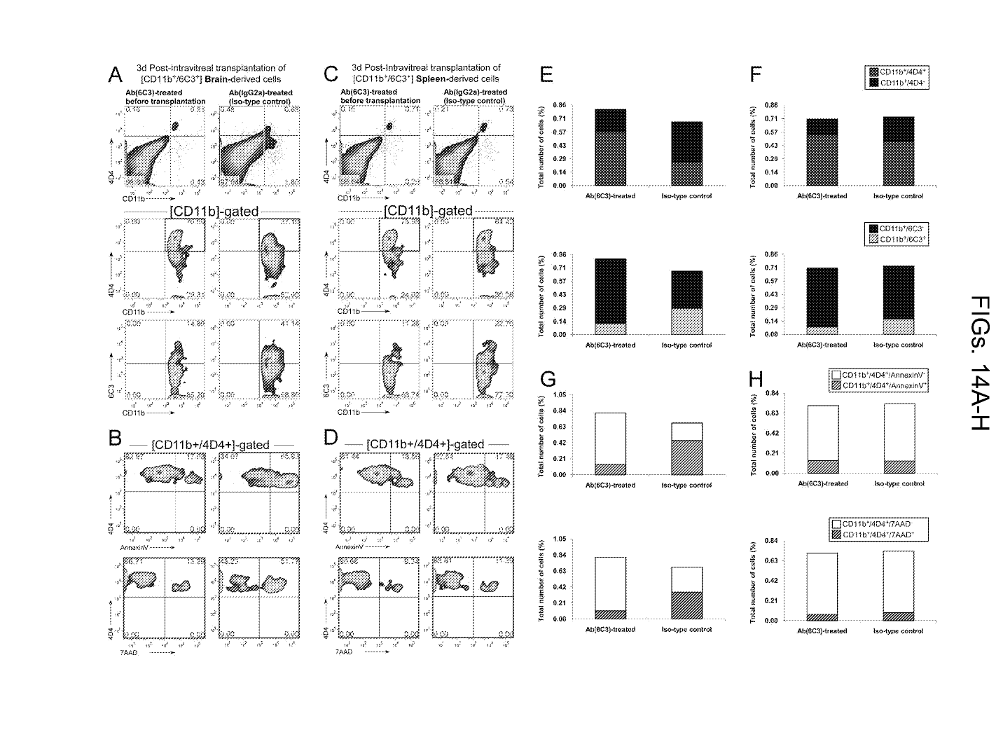

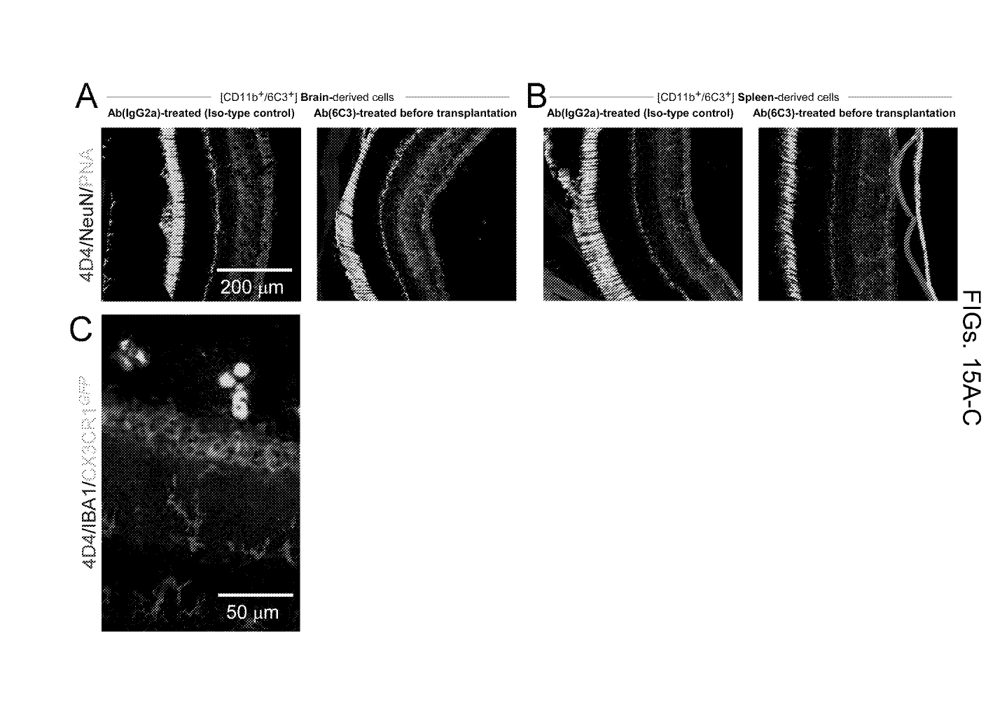

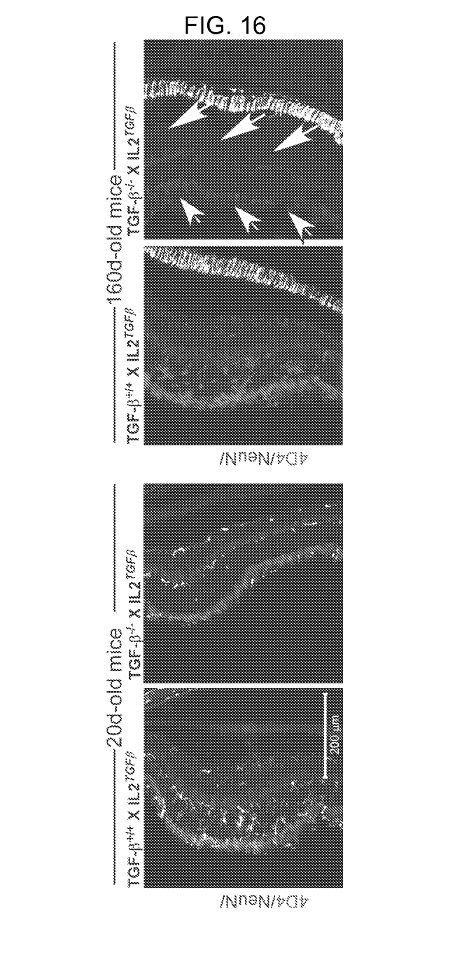

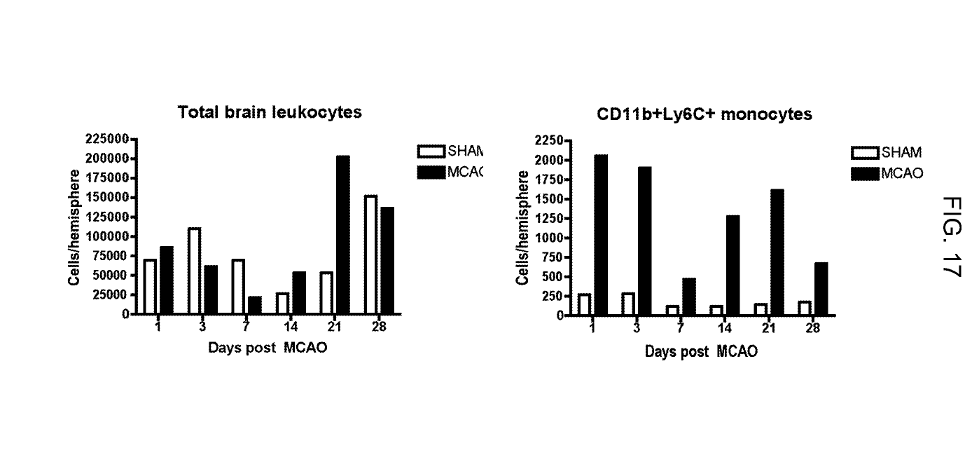

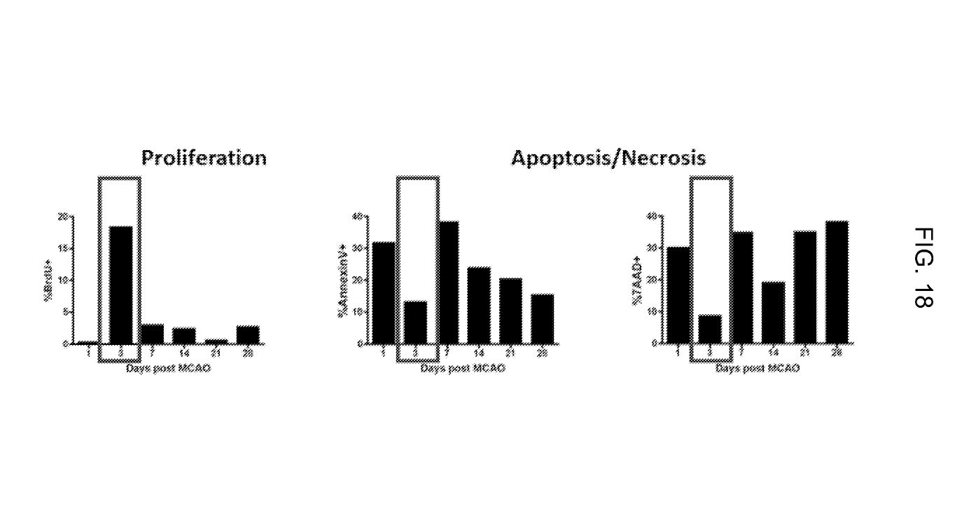

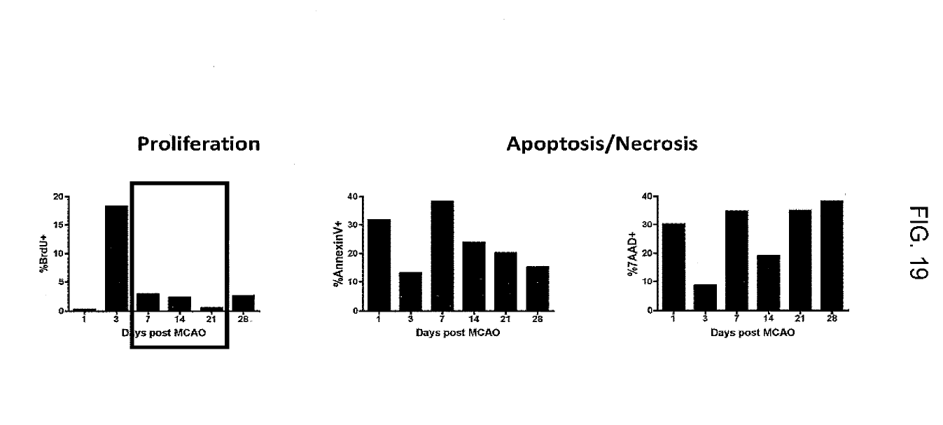

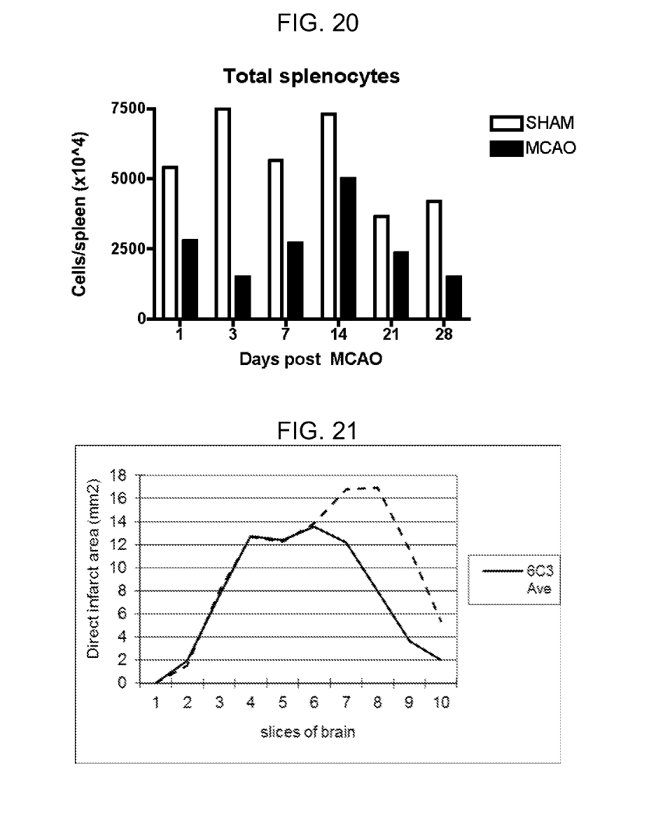

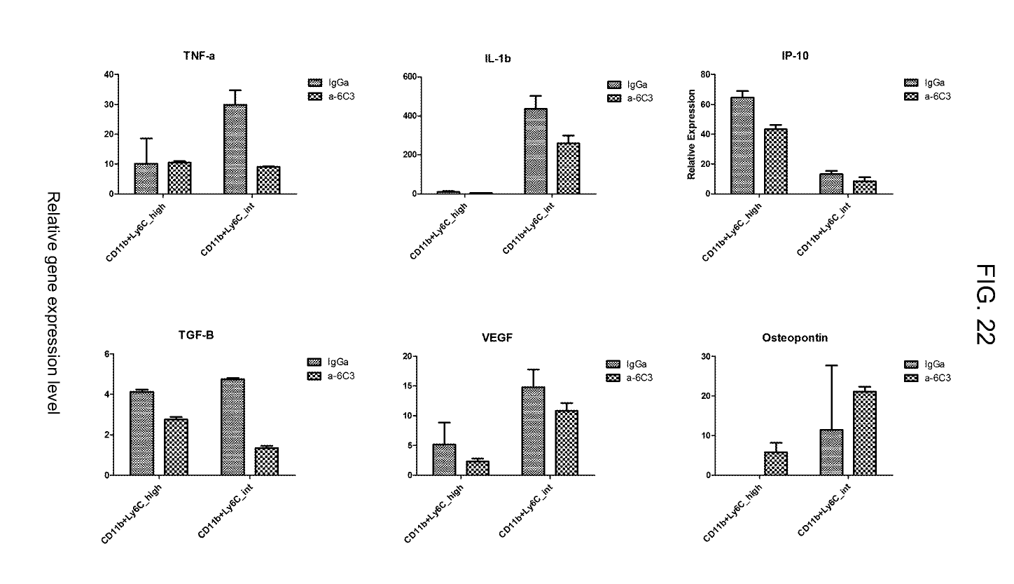

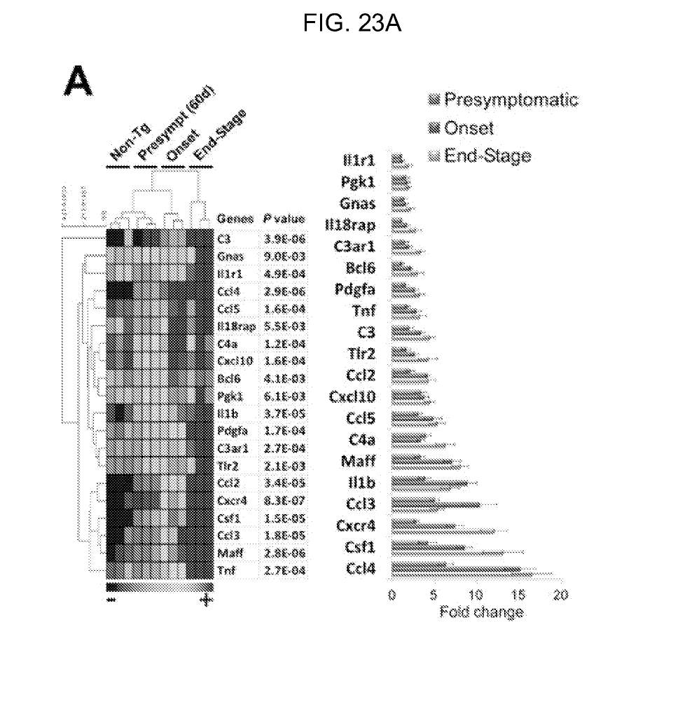

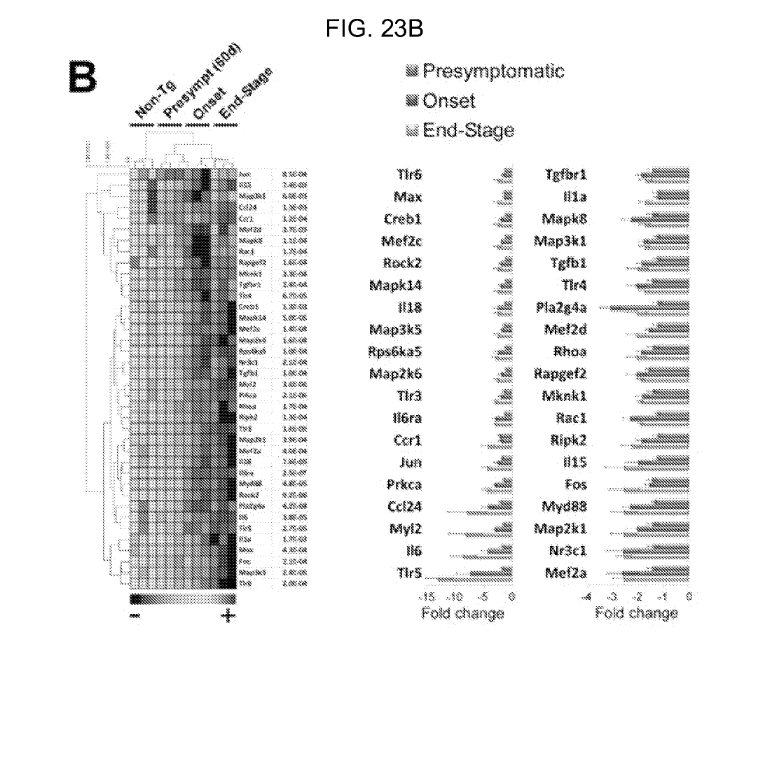

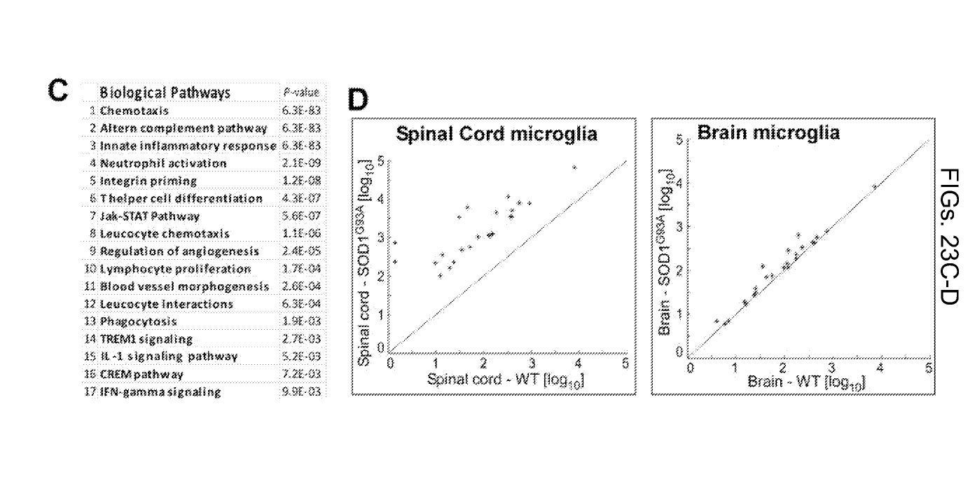

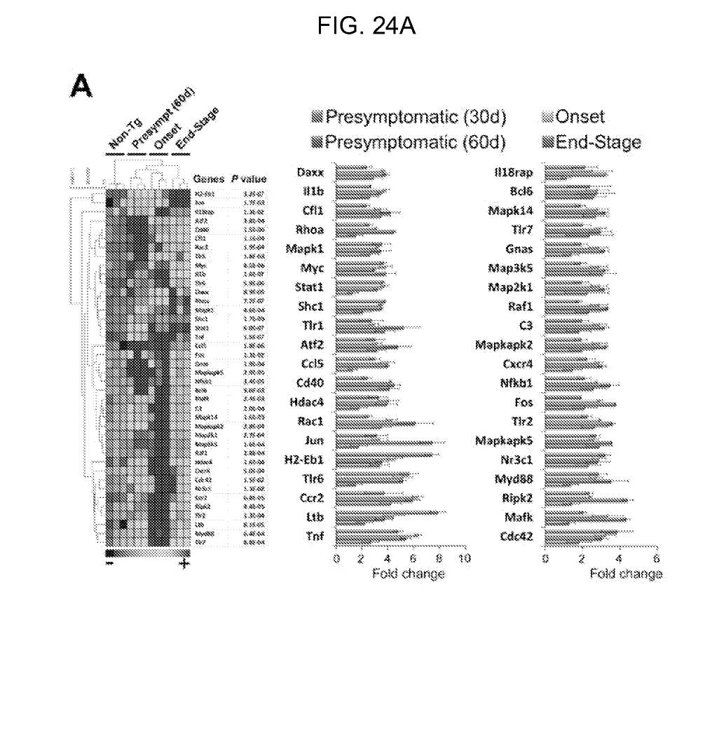

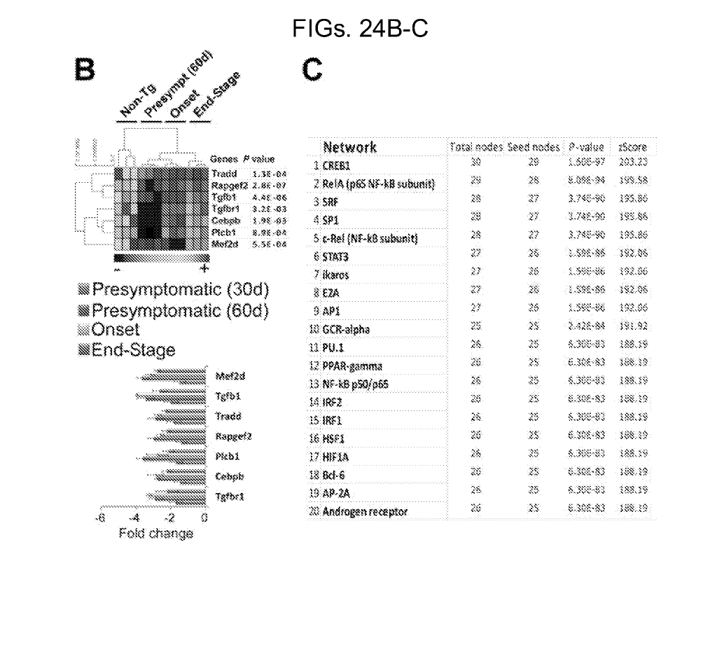

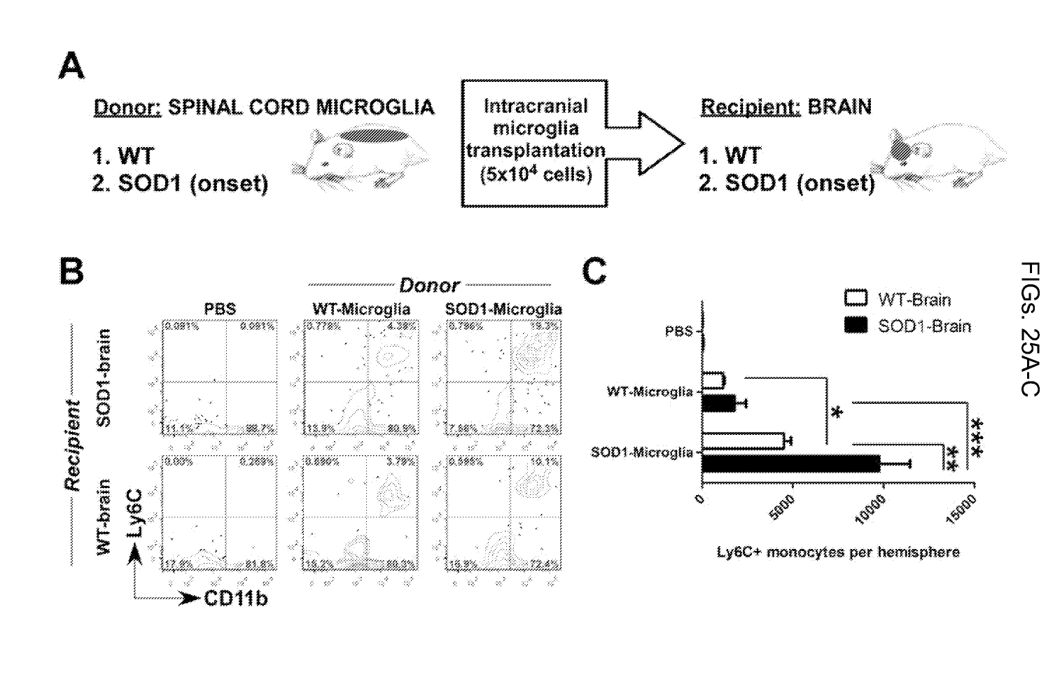

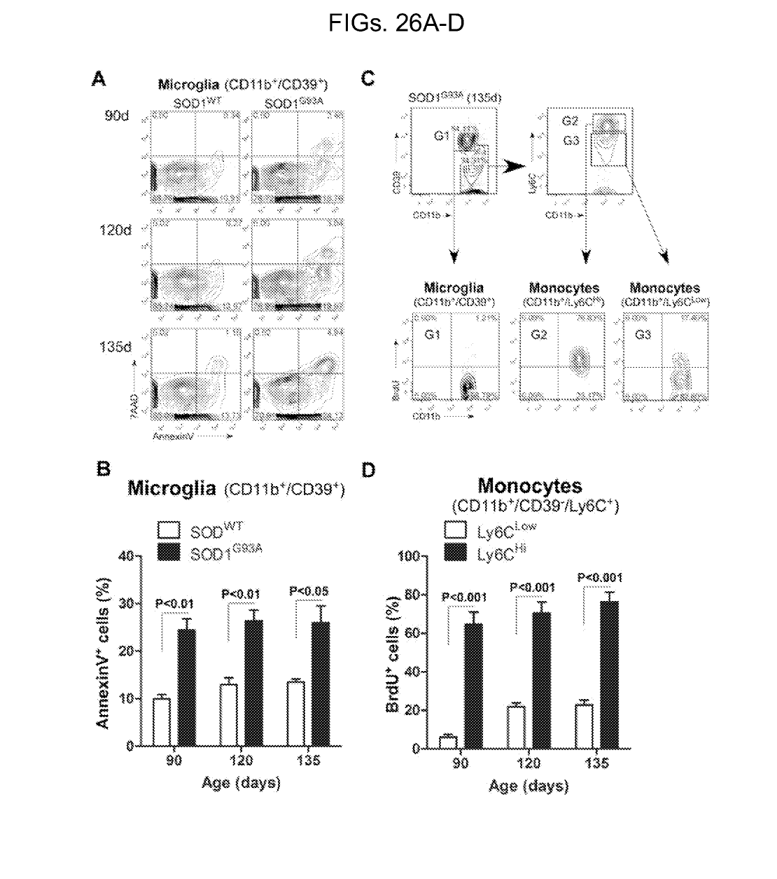

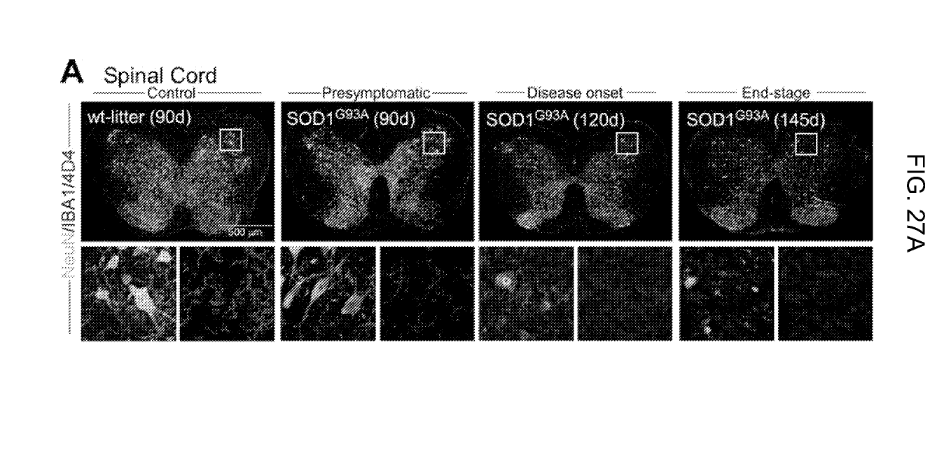

6C3 mAb recognizes inflammatory Ly6C^{Hi} monocytes that originated in periphery and recruited during disease progression in EAE mouse models (FIG. 8). Moreover, during disease progression in EAE, Ly6C expression is significantly upregulated in recruited monocytes in the CNS, PBMCs and splenic monocytes. It was hypothesized that anti-6C3 mAb treatment would target inflammatory monocytes and may change disease progression and has a therapeutic value in autoimmune disease and diseases of the brain associated with recruitment of Ly6C+ inflammatory monocytes. To test this hypothesis, EAE mice were treated with anti-6C3 mAb systemically (ip; 100 ug/injection each 2nd day). Anti-6C3 mAb injected at the onset and peak of the disease (11, 13 and 15 days) delayed the onset and significantly attenuates the clinical score of EAE mice (FIG. 11). In addition, in experiments using the eye as a target for nervous system damage, transplantation of Ly6C^{Hi}-sorted cells from the spinal cord of EAE-mice at the peak of disease to the naïve eye induced 4D4+ microglial apoptosis. This was abrogated when Ly6C^{Hi} cells were treated with anti-6C3 mAb before transplantation (see FIG. 13 below). These results show that Ly6C^{Hi} cells are pathogenic and this can be reversed by anti-6C3 mAb treatment in other models besides ALS.

### Example 8

### Increase in Peripheral Inflammatory Monocytes Recruitment Leads to Retinal Ganglion Cells Loss in Aged Mouse Eyes

When sections of the eyeballs obtained from old (24 months-old) CX3CR1-GFP chimera mice were examined and different regions of the neurosensory retina analyzed, the presence of multiple CX3CR1-GFP peripheral monocytes was accompanied by the disappearance of retinal ganglion cell layer (FIG. 12). This led to the hypothesis that peripheral monocytes play an important role in the loss of retinal ganglion cells that is a characteristic feature of glaucoma.

### Example 9

### Induced Recruitment of Ly6C+Peripheral Inflammatory Monocytes in an Animal Model of Glaucoma

Based on the findings above (see, e.g., Example 8), the D2 glaucoma model was used. Analysis of the number of indigenous microglia and peripheral monocytes in retina of young (8 weeks-old wild type), old (8 months-old wild type) and glaucoma (8 months-old D2) mice showed a decrease in indigenous microglia (4D4+) cells in old as well as in D2 mice. Moreover, it also revealed an increase in number of the peripheral detrimental inflammatory monocytes (6C3+) in the retina of D2 mice. Comparison between optic nerves of old and D2 mice revealed noticeable reduction in the amount of CD11b+ cells, marked decrease of indigenous microglia (4D4+) cells and increase of peripheral inflammatory monocytes (6C3+) in D2 mice. Apoptosis and necrosis in the optic nerve were also more prominent in D2 mice (FIG. 13). These results demonstrated that in the D2 glaucoma model there is a decrease of indigenous microglia associated with the induced recruitment of peripheral inflammatory monocytes.

### Example 10

### Brain Derived Peripheral Recruited Monocytes (CD11b+/Ly6CHi) are Cytotoxic to the Retinal Indigenous Microglia Cells

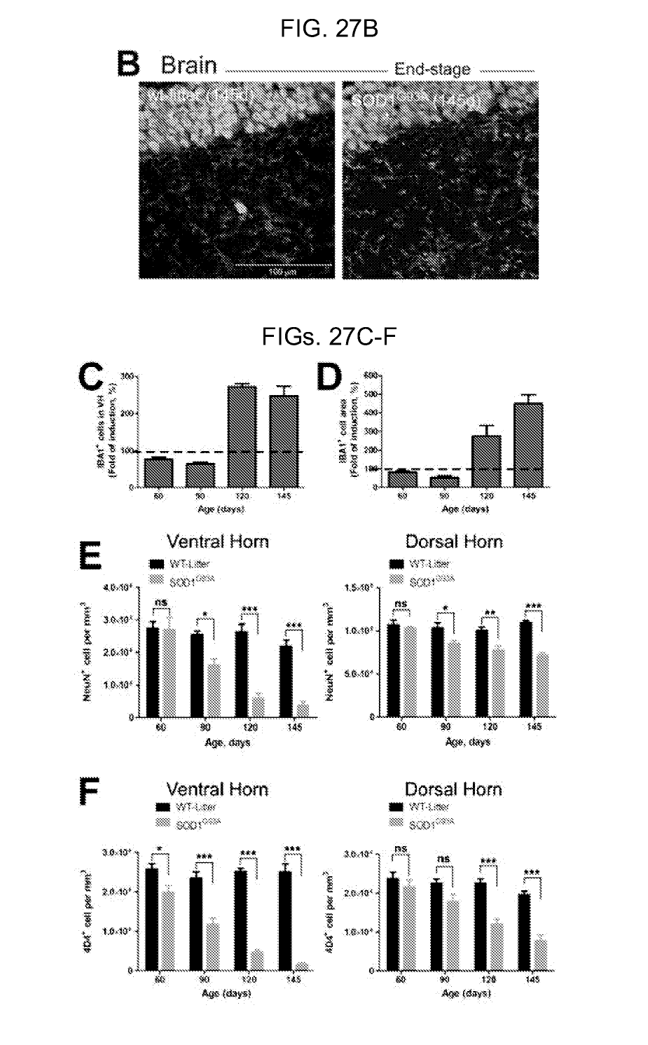

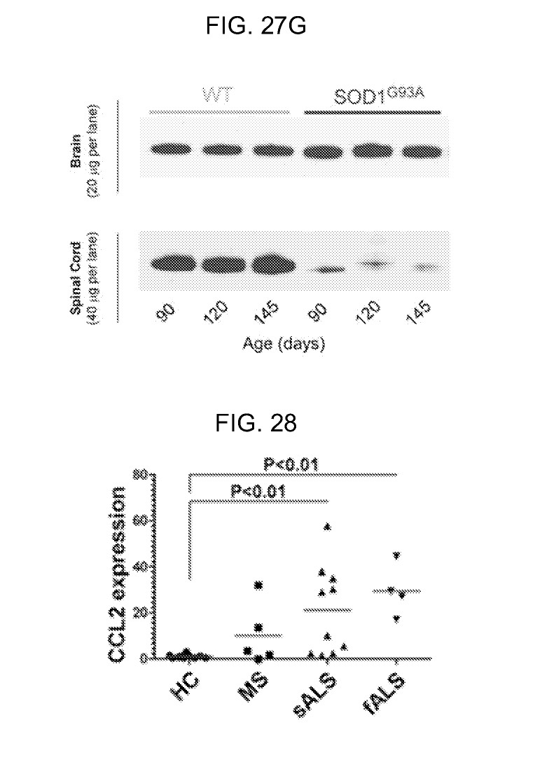

In order to evaluate the effect of Ly6C+ cells on indigenous microglia, CD11b+ cells were sorted from the brains and spleens of EAE-induced 8 week-old wild type mice at the peak of the disease. Then both brain-derived and spleen derived CD11b+ cells were sorted for CD11b+/Ly6C+ cells. After the sorting, both brain-derived nd spleen-derived CD11b+/Ly6C+ cells were divided into two subgroups. The first group of the cells was pre-treated with anti-6C3 antibody and the second subgroup of cells was treated with Ab (Ig2a) as iso-type control before transplantation. Thereafter, these brain-derived and spleen-derived CD11b+/6C3+ cells were transplanted intravitreally to 10 week-old wild type mice. The animals were sacrificed three days after the transplantation and FACS analysis of indigenous microglia and peripheral monocytes in the retina of transplanted animals was performed (FIGS. 14 and 15). There was a significant reduction of indigenous microglia (4D4+/CD11b+) and significant reduction of peripheral inflammatory monocytes (CD11b+/Ly6C+) infiltration when brain-derived CD11b+/6C3+ cells were treated with anti-6C3 antibodies before the implantation. These results were observed only in brain-derived CD11b+/Ly6C+ group and not in spleen-derived CD11b+/Ly6C+ group, where there was no difference in microglia loss. These results demonstrate that anti-6C3 treatment has a neuroprotective effect on indigenous microglia and retinal ganglion cells in an animal model of glaucoma.

### Example 11

### Deficiency of TGFbeta in the CNS Results in Widespread Microglial Loss Accompanied by Increased Recruitment of Ly6C+ Peripheral Monocytes and Retinal Ganglion Cell Loss in the Eye

A new mouse model lacking microglia in the CNS was generated. This model is based on previously described ko-TGF-beta mice (Brionne et al., Neuron40:1133-1145, 2003) crossed with TGF-beta T cell-transgenic mice (Carrier et al., J Immunol. 178(1):179-185, 2007). These mice are specifically deprived of TGF-beta in the CNS, but not in periphery. Histological examination revealed no abnormalities of peripheral organs, however, deficiency of TGF-beta in the CNS results in a widespread microglial loss at very young age (<20 days) (see previous section 2.2). In chimera TGF-beta−/−xIL2TGF-beta mice, transplanted with wt BM-derived cells from tg mice expressing GFP under CX3CR1 at age of 6 weeks, at the end-stage (150-160d), immunohistochemical analysis revealed massive infiltration of GFP+ monocytes co-expressing IBA1 (shares both microglia and monocytes identity) which are not co-express 4D4. Importantly, the majority of these cells were positive for 6C3, indicating that with disease progression, 6C3 is upregulated on peripheral monocytes. No indigenous microglia (IBA1+/4D4+/GFP−) were detected, but peripheral monocytes (IBA1+/4D4−/GFP+) were detected. In addition, in the eye of this mouse, widespread microglia and retinal ganglion cell loss was observed (FIG. 16).

### Example 12

### Neuroprotective Effect of Anti-Ly6C Antibody in Brain Ischemia

The role of these inflammatory monocytes in stroke was evaluated in a middle cerebral artery occlusion (MCAO) mouse model. The MCAO was induced for 1 hour in 8-10 week old B6 male wild type mice using previously described methods (see, e.g., Liu and McCullough, J Biomed Biotechnol. 2011;2011:464701). A sham operated group was used as a control. The post MCAO mortality rate was 30-50%. Animals were sacrificed at 1, 3, 7, 14, 21, or 28 days (n=4/timepoint) and monocytes were isolated from the brain, spleen and blood. FACS Analysis was used to detect immune cell type markers and to measure proliferation and apoptosis. FACS sorting was used to isolate cells for gene expression analysis by RT-PCR.

The results showed a biphasic recruitment of CD11b+Ly6C+ monocytes to the ischemic brain hemisphere following MCAO, with an initial peak at days 3 and a subsequent peak at days 14-12 (see FIG. 17). The “early” CD11b+Ly6C+ monocytes in the ischemic brain displayed enhanced proliferation and reduced cell death at d3 post MCAO (FIG. 18). Later, CD11b+Ly6C+ monocyte frequency in the ischemic brain increased between d7 and d21 despite minimal proliferation (FIG. 19). Gene expression analysis of the CD11b+Ly6C+ monocytes showed that at both the early and late time points, the cells expressed TNF-a and IL-1B, and early cells express VEGF mRNA, while late cells express CCR2.

To determine the origin of the “late” CD11b+Ly6C+ monocytes, splenic size and monocyte populations were evaluated. A significant reduction in spleen size was seen following MCAO (FIG. 20), and biphasic decreases in splenic levels of CD11b+Ly6C+ monocytes were seen that paralleled the increases in CD11b+Ly6C+ monocytes in the brain. The reductions in splenic CD11b+Ly6C+ monocytes was not related to cell death, but rather appeared to part of a non-selective global mobilization of splenocytes following MCAO.

In splenectomized animals, there was a significant reduction in the Ly6C+ monocyte recruitment in the ischemic brains 24 h post R-MCAO.

To determine whether anti-Ly6C antibodies would exert a protective effect in this model, 100 ug of antibody was administered on Day 0 (IV; immediately following MCAO-reperfusion), Day 1 (IP), Day 2 (IP) followed by TTC staining at Day 3. An isotype control (IgG2b) was used in the reference group. The results, shown in FIG. 21, indicate that anti-Ly6C (6C3) treatment during the acute stage of cerebral ischemia reduces infarct size and may improve survival following MCAO.

### Example 13

### Anti-Inflammatory Effect of Anti-Ly6C Antibody in Healthy Animals

To determine what effect, if any, administration of anti-Ly6C antibody would have on inflammation in healthy subjects, expression of TNF-a, IL-1b, IL-6, IL-10, TGF-B, Osteopontin, VEGF1, and IP-10 was measured in wild type animals who had been administered anti-Ly6C antibody or IgGa isotype control on days 1, 3, and 5; the animals were sacrificed on day 6 and expression levels were measured using RT-PCR/Taqman gene expression analysis.

The results, shown in FIG. 21, demonstrated that anti-Ly6C (6C3) antibody treatment of wild type animals induced downregulation of TNF-a, IL-1B, and TGF-B mRNA expression by CD11b+Ly6Cintermediate monocytes in the spleen. There were no detectable levels of IL-6, IL-10 in either group.

### Example 14

### Activation of the Chemotaxis Pathway in CD39⁺ Resident Microglia in the Spinal Cord But Not in the Brain of SOD1 Mice

The ability to distinguish infiltrating monocytes from resident microglia allowed expression profiling of CD11b⁺/CD39⁺ microglia isolated from the spinal cord and brains of SOD1⁺ mice at different stages of disease. The Nanostring nCounter gene expression analysis system (NanoString nCounter, Seattle, Wash.), which is more sensitive than microarrays, similar in accuracy to real-time PCR, and more scalable than real-time PCR or microarrays in terms of sample requirements (Guttman et al., Nature 477:295-300 (2011); Malkov et al., BMC Research Notes 2:80 (2009); Kulkarni, “Digital multiplexed gene expression analysis using the NanoString nCounter system.” In: Current Protocols in Molecular Biology. Ausubel et al., Eds. Chapter 25:Unit25B 10 (2011)), was used. Nanostring detection does not require conversion of mRNA to cDNA by reverse transcription or the amplification of the resulting cDNA by PCR (Geiss et al., Nat Biotechnol 26:317-325 (2009)) and allows expression analysis of up to 800 genes from rare cells (3,000) which is perfectly suited for analysis of the limited number of cells infiltrating the CNS. Out of 179 inflammation-related genes measured by quantitative nCounter, 20 were upregulated (FIG. 23A) and 38 were downregulated relative to non-transgenic wild type mice in spinal cord CD39⁺ resident microglia (FIG. 23B). Microglia had prominent expression of genes related to chemotaxis (e.g., CCL2, CCL3, CCL4, CCLS, CXCR4 and CXCL10). TGFbeta1 and TGFbeta1 receptor were among the downregulated genes. Biological network analysis (MetaCoreTM, GeneGo Inc., St Joseph, Mich., USA) identified activation of inflammatory pathways with the most significant being chemotaxis (FIG. 23C). The expression of these genes was observed one month prior to symptom onset and was observed in the spinal cord, but not in the brain (FIG. 23D).

### Example 15

### Ly6C^{Hi} Monocytes in the Spleen Exhibit a Pro-Inflammatory Profile Two Months Prior to Clinical Disease Onset and During Disease Progression in SOD1 Mice

The gene expression profile of Ly6C^{Hi} monocytes isolated from the spleen of SOD1 mice was examined at one and two months prior to clinical disease onset and during disease progression. A pronounced pro-inflammatory profile was seen at all timepoints (FIG. 24A). Of 179 inflammation related genes measured by nCounter, 40 were upregulated relative to non-transgenic wild type mice. Seven genes that were downregulated in Ly6C^{Hi} cells were also identified including the anti-inflammatory cytokine TGFbeta1 and TGFbeta1 receptor (FIG. 24B). Biological network analysis (MetaCore™ GeneGo) demonstrated the most significantly affected pathways related to inflammatory responses, which included CREB1, NF-kappaB, PU.1 and PPARgamma (FIG. 24C). These pathways have been shown to play an important role in both monocyte activation and differentiation (10-12). Thus, the gene expression profiling demonstrates an activated pro-inflammatory Ly6C^{Hi} monocyte population in the peripheral immune compartment of SOD1 mice that can be observed two months prior to disease onset.

### Example 16

### Ly6C^{Hi} Inflammatory Monocytes Infiltrate the Spinal Cord with Disease Progression in SOD1 Mice

CD11b⁺/Ly6C monocytes and CD11b⁺/CD39⁺ microglia were measured in the CNS of SOD1 mice during disease progression. As noted above (Example 3, FIG. 3A), in wild type mice, 98% of CD11b⁺ cells were CD39 and 1-2% were Ly6C⁺ in both spinal cord and brain. In SOD1 mice with end-stage disease (135 days), 31% of CD11b cells in the spinal cord were Ly6C⁺ and there was a decrease in the number of CD39⁺ cells (22%). No changes in Ly6C⁻ cells or in CD39⁺ cells were observed in the brains of SOD1 mice (FIG. 3A). The changes seen in the spinal cord but not the brain are consistent with the changes in inflammatory gene expression in microglia from spinal cord but not brain (FIG. 3C). Furthermore, these findings suggest a relationship between the recruitment of Ly6C cells and the areas of CNS damage in SOD1 mice.

The percentage of CD11b⁺/Ly6C monocytes and CD11b⁺/CD39⁺ microglia were also quantified in the CNS over time. There was an increase in Ly6C⁺ monocytes which began at 60 days of age (one month before disease onset) and increased as the disease progressed (FIGS. 3B and C). At 120d and 135d, respectively, the proportion of Ly6C monocytes and myeloid cells significantly increased compared with the age 135d wild type mice (P<0.01 and P<0.001, respectively), whereas CD39⁺ microglia significantly decreased (P<0.01 and P<0.001, respectively). No contribution of myeloid subsets was detected in brains of SOD1 mice (FIG. 3B). No Ly6C⁺ monocytes were detected in the spinal cord at 30 days of age, even though they had increased expression of inflammatory genes at this time (FIG. 3A). As shown in FIG. 3D, Ly6C expression is upregulated with disease progression and CD39⁺ microglia remain negative for Ly6C, which is consistent with the observation that CD39+ and Ly6C+ represent non-overlapping CD11b populations (See, e.g., FIGS. 1A-B and 2A-E).

CCL2 interacts with CCR2 receptors on the surface of Ly6C^{Hi} monocytes and is required for the recruitment of Ly6C^{Hi} monocytes to areas of inflammation (Kim et al., Immunity 34:769-780 (2011); Mildner et al., Nat Neurosci 10: 1544-1553 (2007), Nahrendorf et al., J Exp Med 204: 3037-3047 (2007)). As shown above, gene profiling revealed an increase in the expression of CCL2 on microglia (FIG. 23A) and CCR2 on Ly6C^{Hi} monocytes (FIG. 24A). This observation was validated using qPCR to measure the kinetics of CCR2 expression in Ly6C^{Hi} splenic monocytes and CCL2 expression in CD39⁺ microglia in the spinal cord over the course of disease. CCR2 is upregulated in splenic Ly6C^{Hi} monocytes both at disease onset and at end-stage disease. This was paralleled by upregulation of CCL2 on CD39⁺ microglia at disease onset. In addition, there was no expression of CCR2 on CD39⁺ microglia or of CCL2 on Ly6C^{Hi} monocytes at any time during the disease course (FIG. 3E). This suggests that expression of CCL2 and other chemokines (FIG. 23A) on microglia plays a role in the recruitment of Ly6C^{Hi} monocytes to the CNS. Of note, CCL2 expression on CD39⁺ microglia decreases at end stage disease (FIG. 3E).

In order to address direct effect of spinal cord microglia on recruitment of Ly6C+ monocytes in SOD1 mice, donor WT or SOD1 spinal cord-derived microglia were transplanted into the brains of recipient WT or SOD1 mice at onset (FIG. 25A). SOD1 spinal cord microglia significantly induce recruitment of Ly6C+ monocytes (FIGS. 25B and C).

### Example 17

### Ly6C^{Hi} monocytes proliferate and CD39⁺ microglia undergo apoptosis in the spinal cord during disease progression in SOD1 mice

To further investigate Ly6C^{Hi} monocytes and CD39⁺ microglia in the spinal cord during the course of disease, cellular proliferation was measured by BrdU and apoptosis was measured by AnnexinV and 7-AAD staining for apoptotic and necrotic cells, respectively. As shown in FIGS. 27A-D, CD39⁺ microglia in the spinal cord undergo apoptosis at all disease stages (FIGS. 26A and B). Concomitant with this, Ly6C^{Hi} monocytes were recruited to the spinal cord and proliferated at all stages of disease (FIGS. 26C and D). These results demonstrate reciprocal changes in these two cell populations during the course of disease. In addition, immunohistochemistry was performed to detect resident microglia in the spinal cord of SOD1 mice during disease progression using our novel unique microglia 4D4 mAb (FIGS. 27A-F). 4D4⁺ microglial loss occurs during disease progression in the spinal cord, but not in the brain of SOD1 mice.

### Example 18

### Peripheral Monocytes (CD14+/CD16−) in ALS Patients Demonstrate Increased Levels of CCL2

Expression of CCL2 in monocytes from ALS patients and healthy controls was evaluated. Blood samples were collected from 24 healthy control donors, 22 patients with sporadic ALS (sALS), 4 patients with familial ALS (fALS) due to mutations in the SOD1 gene, and 8 relapsing-remitting MS patients. All four fALS patients carried the SOD1 mutation, with specific mutations, including A10G, L113T, A4V, and L9V. Blood was drawn by a study phlebotomist using standard equipment and collected in lithium heparin tubes. Samples were transported to the lab for cell separation within 4 hours of collection. Cells were then frozen until use.

Demographic information for study participants is shown in Table 1. ALSFRS-R: revised ALS Functional Rating Scale; SD: Standard Deviation; sALS: sporadic ALS; fALS: familial ALS

TABLE 1
ALS Patients Donating Blood

| | Disease Type | |
|---|---|---|
| | sALS | fALS |
| Characteristic | | |
| Number of patients | 18 | 4 |
| Disease duration (months) +/− SD* | 30.2 +/− 24.8 | 51.0 +/− 46.2 |
| Age in Years +/− SD | 58.8 +/− 10.8 | 56.3 +/− 8.3 |
| Percent Male | 59% | 75% |
| Mean ALSFRS-R | 34.3 | 37.8 |
| Site of Disease Onset | | |
| Bulbar | 18% | 0% |
| Cervical | 55% | 0% |
| Lumbar | 27% | 100% |
| Unknown | 0% | 0% |

*Disease onset taken as the first of the month for subjects who could identify the month, but not the day of disease onset.

Fresh peripheral blood mononuclear cells were obtained by Ficoll density-gradient centrifugation. CD14+/CD16− and CD14+/CD16+ monocyte subsets stained with mouse anti-human CD14-PE and CD16-PeCy7 (BD Pharmingen) were sorted with a FACSAria (BD Biosciences). The sorted cells were further prepared for the RNA isolation protocol indicated below.

To assess mRNA expression in human CD14+/CD16− and CD14+/CD16+ monocyte subsets, total RNA was isolated and analyzed by real-time qPCR using specific primers for selected mRNAs and miRNAs all purchased from Applied Biosystems. All qRT-PCRs were performed in duplicate or triplicate, and the data are presented as mean±standard deviations (S.D.).

The results showed that CCL2 levels were significantly upregulated in ALS patients as compared to healthy controls (see FIG. 28).

### References

1. Alexianu et al., 2001. Neurology 57: 1282-9
2. Hall et al., 1998.. Glia 23: 249-56
3. Chiu et al., 2008. Proc Natl Acad Sci USA 105: 17913-8
4. Beers et al., 2006. Proc Natl Acad Sci USA 103: 16021-6
5. Boillee et al., 2006. Science 312: 1389-92
6. Beers et al., 2008. Proc Natl Acad Sci U S A 105: 15558-63
7. Stalder et al., 2005. J Neurosci 25: 11125-32
8. Simard et al., 2006. Neuron 49: 489-502
9. El Khoury et al., 2007. Nat Med 13: 432-8
10. Rezai-Zadeh et al., 2009. J Neuroimmune Pharmacol 4: 462-75
11. Frenkel et al., 2005. J Clin Invest 115: 2423-33
12. Lincecum et al., 2010. Nat Genet 42: 392-9
13. Saederup et al., 2010. PLoS ONE 5: e13693
14. Boring L et al., 1998. Nature 394: 894-7
15. Tacke et al., 2007. J Clin Invest 117: 185-94
16. Serbina and Pamer EG. 2006. Nat Immunol 7: 311-7
17. Tsou et al., 2007. J Clin Invest 117: 902-9
18. Auffray et al., 2007. Science 317: 666-70
19. Cardona et al.,. 2006. Nat Neurosci 9: 917-24
20. Mack et al., 2001. J Immunol 166: 4697-704
21. Mildner et al., 2008. J Immunol 181: 2713-22
22. London et al., 2011. J Exp Med 208: 23-39
23. Offner et al., 2006. J Immunol 176: 6523-31
24. Swirski et al., 2009. Science 325: 612-6
25. Banerjee et al., 2008. PLoS ONE 3: e2740
26. O'Connell et al., 2007. Acad Sci U S A 104: 1604-9
27. O'Connell et al., 2010. Immunity 33: 607-19
28. Lu et al., 2009. Immunity 30: 80-91
29. et al., 2009. Clin Cancer Res 15: 2281-90
30. Ceppi et al., 2009. Proc Natl Acad Sci U S A 106: 2735-40
31. Asano et al., 2011. Immunity 34: 85-95
32. Ponomarev et al., 2011. Nat Med 17: 64-70
33. Baraniskin A et al., 2011. Blood 17(11):3140-6.
34. Aarum et al., 2003. Proc Natl Acad Sci USA 100: 15983-8
35. Moreau et al., 2005. Neurology 65: 1958-60

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating a subject suffering from a condition selected from the group consisting of amyotrophic lateral sclerosis (ALS), stroke, and glaucoma, the method comprising administering to the subject an effective amount of a compound that binds to Chemoattractant Cytokine Receptor 2 (CCR2) or Chemokine (C—C motif) Ligand 2 (CCL2).

2. A method of reducing inflammation in a subject suffering from a condition selected from the group consisting of ALS, stroke, and glaucoma, the method comprising administering to the subject an effective amount of a compound that binds to CCR2 or Chemokine (C—C motif) Ligand 2 (CCL2).

3. The method of claim 1, wherein the subject is suffering from ALS.

4. The method of claim 1, wherein the subject is suffering from glaucoma.

5. The method of claim 1, wherein the subject is suffering from a stroke

6. The method of claim 1, wherein the compound is a small molecule inhibitor of CCR2 or CCL2.

7. The method of claim 1, wherein the compound is an antibody or antigenic fragment thereof that binds to CCR2 or CCL2.

8. The method of claim 7, wherein the antibody is a CCR2 binding monoclonal antibody or CCR2-binding fragment thereof, or a CCL2 binding monoclonal antibody or CCL2-binding fragment thereof.

9. The method of claim 7, wherein the antibody is a human, humanized or chimeric antibody.

10. The method of claim 2, wherein the subject is suffering from ALS.

11. The method of claim 2, wherein the subject is suffering from glaucoma.

12. The method of claim 2, wherein the subject is suffering from a stroke

13. The method of claim 2, wherein the compound is a small molecule inhibitor of CCR2 or CCL2.

14. The method of claim 2, wherein the compound is an antibody or antigenic fragment thereof that binds to CCR2 or CCL2.

15. The method of claim 14, wherein the antibody is a CCR2 binding monoclonal antibody or CCR2-binding fragment thereof, or a CCL2 binding monoclonal antibody or CCL2-binding fragment thereof.

16. The method of claim 14, wherein the antibody is a human, humanized or chimeric antibody.
